(19) United States
(12) Patent Application Publication
(10) Pub. No.: US 2023/0105011 A1
(43) Pub. Date: Apr. 6, 2023

(54) STRATIFYING RISK OF MALIGNANCY IN INDETERMINATE THYROID NODULES AND IMMUNO-GENOMIC MARKERS FOR EARLY DETECTION OF THYROID CANCER

(71) Applicant: The University of Toledo, Toledo, OH (US)

(72) Inventors: Shahnawaz Imam, Toledo, OH (US); Juan C. Jaume, Toledo, OH (US)

(73) Assignee: The University of Toledo, Toledo, OH (US)

(21) Appl. No.: 17/790,288

(22) PCT Filed: Jan. 4, 2021
(86) PCT No.: PCT/US2021/012057

§ 371 Date: Jun. 30, 2022

Related U.S. Application Data
(60) Provisional application No. 62/956,769, filed on Jan. 3, 2020.

(51) Int. Cl. G01N 33/574; G01N 1/14

(52) U.S. Cl. CPC G01N 33/57407; G01N 1/14; G01N 2333/52; G01N 2333/70514; G01N 2333/70517

(57) ABSTRACT

Methods for extracting and analyzing a sample, and methods for predicting or diagnosing thyroid cancers and disorders, are described.

### RELATED APPLICATIONS

This application claims priority to U.S. Ser. No. 62/956,769 filed under 35 U.S.C. § 111(b) on Jan. 3, 2020, the disclosure of which is incorporated herein by reference in its entirety.

### STATEMENT REGARDING FEDERALLY SPONSORED RESEARCH

This invention was made with no government support. The government has no rights in this invention.

### BACKGROUND

Thyroid cancer is the most common endocrine cancer globally, with an increasing incidence from 2.4 to 9.4% annually over the last three decades. Thyroid cancer accounts for 3.4% of all new cancer cases in the United States, and 57,000 cases were reported in the year 2017. Thyroid cancer is predicted to become the third most common cancer in women at the end of 2021. The total estimated cost of thyroid cancer treatment in the United States is around $1.5 billion annually. The incidence rate of thyroid cancer is three to four times more frequent in females as compared to males; the cause of this gender difference is still unknown.

Although several clinical practice guidelines to detect/manage thyroid nodules are available, a great deal of controversy still exists towards the optimal approach for diagnosis, in spite of high-resolution ultrasonography of the neck, as well as thyroid fine needle aspiration (FNA) biopsy technique.

It has been shown that in the Euthyroid Hashimoto Thyroiditis condition (EHT), there is about a 48% chance of thyroid cancer. However, there is still not a very accurate predictive marker for early detection of thyroid cancer in Hashimoto's. A similar problem has been observed in the condition known as follicular lesion of undetermined significance (FLUS) or atypia of undetermined significance (AUS). The treatment recommendation typically comes after commercially available genetic testing which only has a suboptimal diagnosis rate as explained below.

A number of molecular and gene mutation diagnostic tests have been developed to diagnose the indeterminate thyroid nodules in fine needle aspiration (FNA) specimens. The positive predictive value of these tests ranges from 40% to 50%, which means that half of the patients going for surgery have a benign module for which surgery was not needed. Thus, tumor gene sequencing by itself is not sufficient to predict thyroid malignancy.

There is a need for more accurate diagnostic tools to predict the chances of having thyroid cancer or for diagnosing thyroid cancer all together.

### SUMMARY

Provided is a method for collecting and analyzing a sample, the method comprising extracting a tissue sample from a patient through a fine needle aspiration (FNA), wherein the tissue sample is extracted from an area comprising fluid within and adjacent to a thyroid nodule; analyzing lymphocytes in the extracted tissue sample; and determining an amount of double negative T cells present in the lymphocytes.

In certain embodiments, the FNA is ultrasound-guided FNA.

In certain embodiments, the area is where the fluid meets the thyroid nodule. In particular embodiments, the fluid comprises blood. In particular embodiments, the fluid comprises immune cells.

In certain embodiments, the method further comprises analyzing the extracted tissue sample for macrophages and NK cells.

In certain embodiments, the method further comprises removing the patient's thyroid if the thyroid nodule is determined to be, or likely to become, cancerous based on the amount of double negative T cells present in the lymphocytes in the extracted tissue sample.

In certain embodiments, the method further comprises profiling the lymphocytes with flow cytometry.

In certain embodiments, the method further comprises analyzing the extracted tissue for chemokines and cytokines using a cytometric bead array or other ultra-sensitive cytokine assay.

In certain embodiments, the method further comprises sequencing of tumor associated lymphoocytic RNA in the extracted tissue sample.

Further provided is a method for diagnosing thyroid cancer, the method comprising obtaining a tissue sample from an area within and adjacent a thyroid nodule in a patient; measuring the amount of double negative T cells in the tissue sample relative to other lymphocytes in the tissue sample; determining whether the thyroid nodule is, or is likely to become, cancerous, wherein a double negative T cell content of greater than 15% in the obtained sample indicates the thyroid nodule is, or is likely to become, cancerous, and a double negative T cell content less than 5% in the obtained sample indicates the thyroid nodule is not, and is not likely to become, cancerous; and removing the patient's thyroid if the thyroid nodule is determined to be, or likely to become, cancerous; or not removing the patient's thyroid if the thyroid nodule is not determined to be, or not likely to become, cancerous.

In certain embodiments, the method further comprises administering a treatment for Hashimoto's disease based on the determination of whether the thyroid nodule is, or is likely to become, cancerous.

In certain embodiments, the tissue sample is obtained through a fine needle aspiration (FNA).

In certain embodiments, the tissue sample is obtained through an ultrasound-guided fine needle aspiration.

In certain embodiments, the area comprises fluid. In particular embodiments, the area is where the fluid meets the thyroid nodule. In particular embodiments, the fluid comprises blood. In particular embodiments, the fluid comprises immune cells.

Further provided is a method for diagnosing thyroid cancer, the method comprising extracting a tissue sample from a patient through a fine needle aspiration (FNA), wherein the tissue sample is from an area comprising fluid within and adjacent to a thyroid nodule, wherein the fluid comprises immune cells; measuring the amount of double negative T cells, macrophages, and NK cells in the tissue sample; and determining whether the thyroid nodule is, or is likely to become, cancerous; wherein a double negative T cell content of greater than 15% in the obtained sample indicates the thyroid nodule is, or is likely to become, cancerous, and a double negative T cell content less than 5% indicates the thyroid nodule is not, and is not likely to become, cancerous.

In certain embodiments, the FNA is ultrasound-guided FNA.

In certain embodiments, the method further comprises removing the patient's thyroid if the thyroid nodule is determined to be, or likely to become, cancerous. In certain embodiments, the method further comprises not removing the patient's thyroid if the thyroid nodule is not determined to be, or not likely to become, cancerous.

Further provided is a method for reverting an M2 phenotype into an M1 phenotype, the method comprising culturing M2 macrophages with NK cells to revert an M2 phenotype into an M1 phenotype.

Further provided is a method for reverting a pro-cancerous M2 phenotype into anti-cancerous M1 phenotype method comprising culturing M2 macrophages with Flagellin to revert the M2 phenotype into an M1 phenotype.

### BRIEF DESCRIPTION OF THE DRAWINGS

FIG. 1: Flow cytometry of resected thyroid samples. Contour plots of lymphocyte specimens from representative patients with papillary thyroid cancer (PTC) and Hashimoto Thyroiditis (Hash) (right), and bar graphs of statistical analysis (left) of untreated (uninduced) lymphocyte specimens gated for CD3 and subsequently sorted for CD4, CD8, DNT, and NKT. DNT stands for double negative, and NKT stands for natural killer, T cells. Black bars represent results for specimens from PTC patients. White bars represent results for specimens from Hashimoto's patients (Hash).

FIGS. 2A-2D: Relationship of Graves Disease (GD), Hashimoto Thyroiditis (HT), and Differentiated Thyroid Cancer (DTC) in patients undergoing thyroidectomy. FIG. 2A shows the study design with benign (BEN) vs. malignant (DTC) outcome as related with either autoimmune (GD and HT with subgroups euthyroid and hypothyroid) or non-autoimmune (Non-AITD). FIGS. 2B-2C show bar graphs for frequency of DTC in each group (FIG. 2B) and Hashimoto's subgroups (FIG. 2C). FIG. 21) shows a bar graph for distribution of DTC based on tumor size.

FIGS. 3A-3D: Flow cytometry analysis of natural killer (NK) cells. Bar graphs of statistical analysis of NK cell present in patient samples (FIG. 3A) and functionality as measured by Interferon gamma (INFO from patients with Euthyroid Hashimoto Thyroiditis (EHT) (n=8) and Graves disease (GD) (n=8) (FIG. 3B) are shown. FIG. 3C shows flow cytometry raw data sample of NK cells in EHT or GD and a bar graph statistical quantification based on production of cytotoxic enzymes Granulysin, Granzyme B, and Perforin. Statistical significance was determined by using t-test: two samples assuming unequal variance. ^{ab} depicts the difference (P<0.05) between the groups.

FIGS. 4A-4B: Macrophage and B cell comparison under induction/stimulation. Macrophages in EHT (n=8) and GD (n=8) before and after induction/stimulation (FIG. 4A) as analyzed by flow cytometry were compared with B cells from the same patients (FIG. 4B) before and after induction/stimulation under high dose of LPS (100 ng/ml) for 54 h.

FIGS. 5A-5D: Intra-thyroidal immune profiling of M1 and M2 macrophage polarization using Flow Cytometry Analysis. Flow cytometry analysis of M1 macrophages (FACS) contour plots (upper panel) of representative patients and Bar graphs (Lower panel) of statistical analysis of leukocyte specimens from patients with Euthyroid Hashimoto Thyroiditis (EHT) (n=8) and Graves disease (GD) (n=8). Leukocyte specimens were gated for CD3−ve and subsequently sorted for macrophages (CD14 and CD68). Macrophages were re-gated for the M1 macrophage activation marker Viz. CCR2, CXCR1, IL12, TNFa, and iNOS; uninduced (FIG. 5A) and induced (FIG. 5B) are shown. Macrophages were again re-gated for the M2 macrophage activation markers Viz. Arginase 1, Dectin 1, and IL10. Uninduced (FIG. 5C) and induced (FIG. 5D) are shown. Statistical significance was determined by using t-test: two samples assuming unequal variance.

FIGS. 6A-6M: NK-Macrophage crosstalk. Peripheral Blood M1-M2 differentiation and quantification of final product. M1 (FIG. 6A) and M2 (FIG. 6B) structural phenotypes are shown. Macrophages were labeled with CD68-PE (red), cytoskeleton stained with phalloidin-Alexa Fluor 488 (green), and nuclei stained with DAPI (blue). Bar graph of proportions of M1 and M2 as well as NK cells in active (NA) or resting (N0) form as quantified by flow cytometry (FIG. 6C). Naïve/resting NK cells were activated by using IL-2 at the dose rate of 50 ng/ml. Macrophages were differentiated from human PBMCs into M1 and M2 macrophages. Differentiated macrophages (M1 and M2) and NK (NA and N0) cells were co-cultured. All the experiments were executed in triplicate and the mean of the three was considered as an individual observation (n=3-6). Autologous co-cultures of M1/M2 macrophage with NA/N0 NK cells were stained with M1/M2 phenotype markers. Phenotypic characterization of differentiated macrophages (M1 and M2) were done using a flow cytometer. Single live cells were gated and subsequently sorted for macrophages (CD68) and re-gated for the M1 macrophages activation marker viz. CCR2, CX3CR1 for surface chemokine and IL-12 and TNFa for intracellular cytokines (FIGS. 6D-6F). In the same autologous co-culture experiment, the macrophages were re-gated for CD68 and gated for M2 macrophage activation marker viz. Arginase 1, Dectin 1, and IL-10 (FIGS. 6G-6I). Again in the same autologous co-culture experiment single cells were gated for NK cell markers (CD56+CD3−) and subsequently sorted for intracellular cytokines and multimeric complexes viz. GZB, IFNg, and Perforin (FIGS. 6J-6L). Statistical significance was determined by using t-test: two samples assuming equal variance. NK cells were co-culture against macrophages at different Effector to Target (E:T) ratios. Resting and activated NK (N0 and NA) cells cytotoxicity against macrophage (M0, M1, and M2) is shown (FIG. 6M). Cytotoxicity was assessed in flow cytometer using CFSE-FITC for alive and 7 Aminoactinomycin D (AAD)-PE for dead cells. ^{AB} depict the difference between the groups within a ratio and ^{ab} depict the difference (P<0.05) among the ratios within a group.

FIGS. 7A-7B: Image showing a common immune infiltration surrounding thyroid cancer (FIG. 7A) and fine needle aspiration (FNA) site adjacent/intersection of nodule and immune microenvironment (FIG. 7B).

FIGS. 8A-8B: Illustration showing crosstalk between NK cells and macrophages in thyroid cancer coexisting with Graves' disease and euthyroid Hashimoto's thyroiditis. Activated NK cells and M1 macrophages induce tumor regression and protect from cancer as in case of Graves' disease (No cancer) while euthyroid HT, low/no NK cells tip the macrophages balance towards M2-dominance may contribute to tumor progression (pro cancerous/cancer) (FIG. 8A). Illustration showing the immunomodulatory role of flagellin, a therapeutic approach for anaplastic thyroid cancers which are densely intermingled with ramified M2 macrophages with long and thin cytoplasmic processes (FIG. 8B).

FIG. 9: Illustration showing DN T cells regulate proliferation and effector function of T cells. Fas-FasL leads to activation-induced cell death, a form of apoptosis induced by repeated TCR stimulation, responsible for the peripheral deletion of activated T cells. Naturally Fas-FasL resistant DN T cells also induce apoptosis to NK cells. Absence of, or a low count of, active NK cells, plus macrophage plasticity, allows macrophage subtype M0 to differentiate to the M2 phenotype (precancerous/cancerous).

FIG. 10: Technology layout illustration. The patients and control samples were collected. The FNA samples were subjected to cellular sorting followed by cellular profiling using flow cytometry and cytokine/chemokine analysis using cytokine beads array/other tissue lysate method. The sorted immune cells were also subjected to RNA transcript analysis. Finally, integration of genomic and transcriptomic analysis along with cellular profiling, and cytokine/chemokine analysis present in the tissue lysate is used to produce a comprehensive biomarker. This data also include the micro RNA (miRNA) analysis and expression profiling (RNA-Seq) of immune cells present in the microenvironment of thyroid cancer to pinpoint the mechanism(s) involved in the onset and/or progression of the disease. This application further establishes the integration of the information derived from transcriptome/meta-analysis of the immune cell genome to the cellular profiling and cytokine/chemokine signals recorded from an FNA lysate.

FIG. 11: Table 3 showing—Differentiated thyroid cancer incidence and pathological features in patients with and without autoimmune thyroid.

FIG. 12: Table 4 showing a summary of the immune cell profiling from thyroid bed microenvironment of Graves and Euthyroid FIT setting.

FIGS. 13A-13K: Proinflammatory cytokines expression of M2 macrophages were comparable to M1 macrophage expression profiles.

FIGS. 14A-14L: Similar results were observed in autologous co-cultures of M0 monocytes with NK cells.

FIGS. 15A-15P: Proinflammatory cytokines expression of M2 macrophages were comparable to M1 macrophage expression profiles.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Nodules or abnormalities in the body are often detected by imaging examinations. However, it is not always possible to tell from imaging tests or biopsies whether a nodule is benign or cancerous. Approximately 20-30% of cytology results from thyroid FNA biopsies fall into one of three indeterminate diagnostic categories: 1) atypia of undetermined significance/follicular lesion of undetermined significance (AUS/FLUS), 2) follicular neoplasm/suspicious for follicular neoplasm (FN/SFN), and 3) suspicious for malignancy (SM).

Conventional predictive markers for thyroid cancer are based on genotype. In contrast, the present disclosure describes markers based on phenotype of the thyroid cancer microenvironment, and the diagnosis is based on the cellular cross talk (phenotypic and genotypic). In accordance with the present disclosure, the thyroid bed microenvironment is profiled using, for example, fine needle aspiration (FNA), and a sample from the microenvironment is used as a predictive, as well as prognostic, marker for thyroid cancer. The integration of the cellular, humoral, molecular, and genetic information is utilized as an early diagnosis marker of thyroid cancer.

Double negative T (DN T) cells are T lymphocytes that express the αβ T cell receptor (TCR) but do not express CD4, CD8, or natural killer (NK) T cell markers. In normal humans, DN T cells exist as a small (˜1-5%) population of lymphocytes in the peripheral blood and lymphoid organs. However, in accordance with the present disclosure, an overabundance of DN T cells in tissue extracted from an area within and adjacent to a thyroid nodule is indicative of the thyroid nodule being, or being likely to become, cancerous. In particular, thyroid cancer is diagnosed or predicted with extraordinary accuracy when DN T cells make up greater than 15% of the lymphocytes present in tissue extracted from an area within and adjacent to an indeterminant thyroid nodule. Furthermore, when DN T cells make up less than 5% of the lymphocytes present in tissue extracted from an area within and adjacent to an indeterminant thyroid nodule, it is indicative of the thyroid nodule not being, and not being likely to become, cancerous.

The present disclosure is based on the functional correlation of immune-cytokines-communication molecules present in the cellular microenvironment of thyroid cancer. Thyroid cancer is diagnosed with good accuracy by utilizing cellular profiling of the cancer microenvironment (i.e., immune cells, their phenotype, and their immuno-genetic and epigenetic changes). Furthermore, genomic and transcriptomic profiling is utilized in combination with cellular profiling for enhanced accuracy of diagnosis.

Cancer microenvironment profiling is the most accurate way to diagnose and assess the status of disease progression. Current diagnostic methods are based on gene sequencing of cancer cells, whereas the present disclosure describes the cellular profiling of the microenvironment (immune cells, their phenotype, and genetic and epigenetic changes) using FNA. Thyroid cancer diagnosis is based on the cellular crosstalk between immune and cancer cells (phenotype and genotype analysis). The present disclosure further establishes an integration of the information derived from transcriptome/meta-analysis of the genome, cellular profiling, and cytokine/chemokine signal analysis, all from FNA of immune cells in the thyroid cancer microenvironment.

In general, the method described herein is based on cellular profiling of the cancer microenvironment of the thyroid cancer bed using a suitable extraction technique such as fine needle aspiration (FNA). An FNA biopsy is generally the most accurate test for evaluating thyroid nodules. FNA removes some cells, in a less invasive procedure involving a hollow needle, from a suspicious area within the body so that the removed cells are examined under a microscope to determine a diagnosis. During a fine needle aspiration biopsy of the thyroid, a small sample of tissue is removed from the thyroid gland. The thyroid gland is located in the front of the neck just above the neckline and is shaped like a butterfly, with two lobes on either side of the neck connected by a narrow band of tissue. In accordance with the present disclosure, FNA is used to extract a tissue sample from an area within and adjacent to a thyroid nodule.

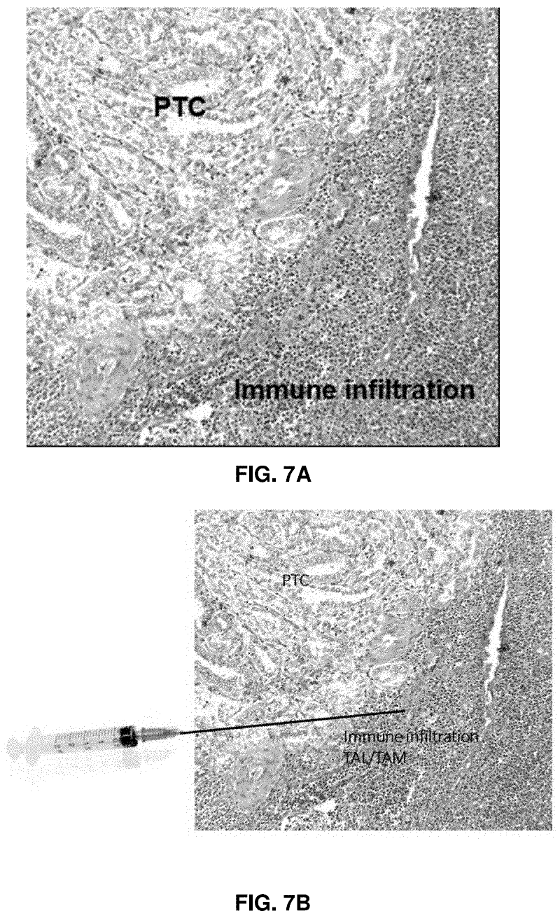

A biopsy is performed under ultrasound guidance. A very thin needle is guided into the thyroid nodule, and a small sampling of cells is aspirated or sucked into the needle. These cells may then be examined under a microscope by a cytologist to make an accurate diagnosis. The sample is obtained through ultrasound-guided FNA (USGFNA). An ultrasound-guided fine needle aspiration biopsy uses sound waves to help locate a nodule or structural abnormality within the thyroid. The needle used is generally a fine-gauge needle, which is smaller in diameter than the needle used in most blood draws (usually a 25 or 27 gauge 1.5 inch needle). The aspiration is done with a needle or with a needle that is attached to a syringe. The syringe is placed in a plastic or metal holder to make it easier to aspirate the cells. Ultrasound is used to guide accurate placement of the needle within or adjacent/intersection to the thyroid nodule (FIG. 7B). It is a minimally invasive procedure of the thyroid gland and is typically performed by a specially trained endocrinologist/radiologist with experience in needle aspiration and ultrasound.

In one non-limiting example method, the neck is first cleansed with antiseptic. Anesthesia given to numb the area may or may not be used. An ultrasound transducer with a small amount of sterile water-soluble gel is placed on the neck over the thyroid area. The endocrinologist/radiologist inserts the needle through the skin under direct imaging guidance, advances it to the site of the thyroid nodule/solid mass-infiltrate interface (FIG. 7B), and aspirates samples of tissue. The ultrasound is used to guide accurate placement of the needle within or adjacent to the thyroid nodule. After the sampling, the needle is removed. New needles are reinserted if additional samples are required. In certain embodiments, several specimens may be needed for a complete analysis.

Image-guided, minimally invasive procedures such as FNA of the thyroid are most often performed by a specially trained endocrinologist/radiologist with experience in needle aspiration and ultrasound. However, this is not strictly necessary.

For the evaluation of the nodule microenvironment, needles are directed to the surroundings of the structural abnormality (i.e., to an area within and adjacent/intersection to the nodule). The extracted material is washed out onto appropriate buffers for subsequent flow cytometry profiling of cells and lysate analysis. A tissue sample is removed for examination under a microscope. The tissue lysate is analyzed for the chemokines and cytokines present in the thyroid tissue lysate using cytometric bead array (CBA), a multiplexed and advanced bead-based immunoassay. A part of the tissue is saved for RNA sequencing. The procedure is less invasive than a surgical biopsy, leaves little to no scarring, and does not involve exposure to ionizing radiation. The procedure also requires little to no special preparation.

In accordance with the present disclosure, markers for thyroid cancer is utilized based on the concept that cancers talk to host immune cells for regulation. Cancers edit a conductive microenvironment for their survival and propagation via immune editing. There is a characteristic population of T cells, known as double negative (DN) T cells, which regulate immune cells in the cancer microenvironment to facilitate cancer cell survival and propagation. Furthermore, in the setting of EHT, the macrophage phenotype is not just different but also has a higher degree of plasticity. The presence of functionally active NK cells and a higher M1/M2 macrophage ratio may also provide a more effective form of tumor immunity. However, in the absence of, or a low count of, active NK cells, macrophage plasticity allows macrophage subtype M0 to differentiate to the M2 phenotype (precancerous/cancerous).

Small non-coding RNAs such as circular RNAs (circRNA), micro RNAs (miRNAs), and the like, have been attributed in the pathogenesis of thyroid cancer. CircRNAs are a promising diagnostic and prognostic biomarker, as they are resistant to exonucleases and are more stable. Very limited information is available regarding the role of circRNAs in thyroid cancer pathogenesis. Sixteen significantly differentiated circRNAs have been identified, out of which twelve circRNAs are upregulated and four are downregulated, in papillary thyroid cancer (PTC). This indicates that circRNA dysregulation may play a role in PTC pathogenesis, and they are useful as biomarkers for thyroid cancer. Another important class of small non-coding RNAs is miRNAs. It is possible that miRNAs, such as miR-21, miR-29b, miR-31, miR-138, miR-139, miR-146, miR-155 miR-204, miR-221, miR-222, and miR-181a, play a role in thyroid cancer. However, their mechanistic details in the thyroid cancer regulation are not fully unraveled. Furthermore, bioinformatics analysis has revealed that one of the downregulated circRNAs (hsa_circRNA_100395) displays interactive ability with two cancer related miRNAs (hsa_circRNA_100395/miR-141-3p/miR-200a-3p network axis). Correlation of circRNA expression with miRNA and mRNA has largely remained unexplored.

Meta-analysis of circRNA, miRNA, and RNA-seq of thyroid samples with cellular profiling and chemokines/cytokines profiling may identify the immunological interactions of the host immune system with circRNA and miRNA. This multi-transcript (RNA/immune cells/humoral mediator approach) may unveil which gene and pathways correlate with the immunological expression patterns before, during, and after the onset of thyroid cancer. A variety of modeling tools may be used to determine which gene/circRNA/miRNA and cellular/molecular parameters are most predictive of thyroid cancer onset and progression. Integrating these multi-transcriptomics datasets allows for the estimation of a multigene/circRNA/miRNA metabolic network, which captures changes in metabolic flow and cooperative or antagonistic functional interactions. Exploring RNAseq datasets provides mechanistic insight as to if and how therapeutic interventions may promote restoration of thyroid homeostasis. The in depth, multi-transcriptome data allows for measuring of the dynamic fluctuation in circRNA/miRNA and cellular and molecular markers longitudinally, providing a large window of opportunity to modify risk factors before and during thyroid cancer.

Information derived from transcriptome/meta-analysis of the genome is integrated to the cellular profiling and the signals recorded from tissue lysate. A functional correlation of differential expression of small noncoding RNAs with immuno-cytokines present in the cellular microenvironment of thyroid cancer is established. The relationship and integration of the genomic and transcriptomic profiling with cellular profiling, and the signals present in the tissue lysate, yields diagnostic markers for early prediction of thyroid cancer with good accuracy.

Modeling the cellular, molecular, and genetic networks of different thyroid diseases allows for determining how these interactions affect the disease onset and progression. Integrating these multi-transcript datasets allows the estimation of metabolic and signaling networks, which also capture the changes in genetic transcriptome, molecular, cellular signatures, and metabolic interactions.

In one non-limiting example embodiment, a tissue sample is extracted from an area within and adjacent to a thyroid nodule through a fine needle aspiration, and the lymphocyte content in the extracted tissue sample is analyzed to determine the proportion of DN T cells present in the lymphocytes. Where DN T cells make up greater than 15% of the lymphocytes present in the extracted tissue sample, it is indicative of the thyroid nodule being, or being likely to become, cancerous. Where DN T cells make up from 5% to 15% of the lymphocytes present in the extracted tissue sample, secondary markers like longitudinal analysis and the integration of a multitude of cellular and humoral ques such as genetic markers is considered to determine the likelihood that the thyroid nodule is, or is likely to become, cancerous. Where DN T cells make up less than 5% of the lymphocytes present in the extracted tissue sample, it is indicative of the thyroid nodule not being, and not being likely to become, cancerous. A suitable treatment option may be taken based on the prediction or diagnosis. For example, if it is predicted that the thyroid nodule is, or is likely to become, cancerous, the patient's thyroid may be removed. If it is predicted that the thyroid nodule is not, and is not likely to become, cancerous, then the patient may forego a thyroid removal.

Notably, whereas previous disclosures regarding DN T cells have involved the study of ex vivo samples of thyroids themselves, the present disclosure provides for the prediction of thyroid cancer based on in vivo conditions in an area within and adjacent to a thyroid nodule as seen through extracted tissue samples.

### EXAMPLES

Certain embodiments of the present invention are defined in the Examples herein. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

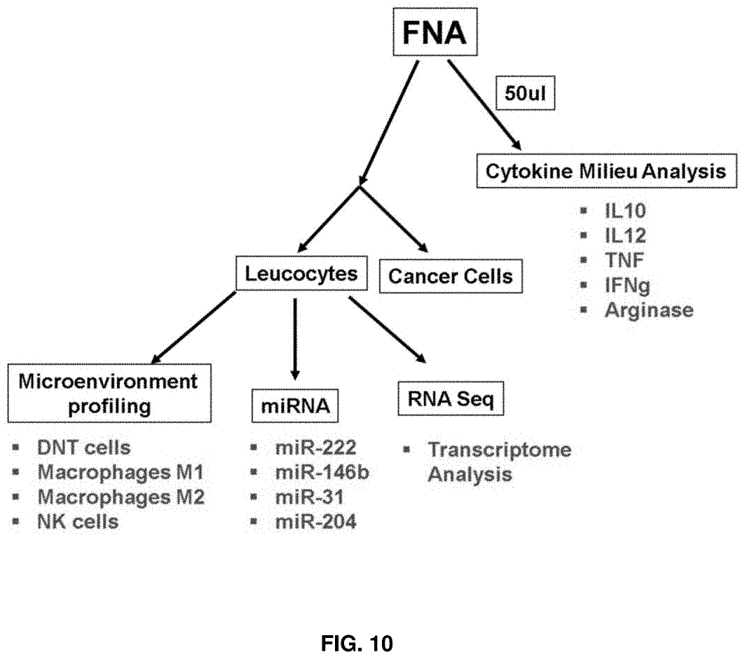

Referring first to FIG. 10, a technology layout is generally illustrated. The patients and control samples were collected. The FNA samples were subjected to cellular sorting followed by cellular profiling using flow cytometry and cytokine/chemokine analysis using cytokine beads array/other tissue lysate method. The sorted immune cells were also subjected to RNA transcript analysis. Finally, integration of genomic and transcriptomic analysis along with cellular profiling, and cytokine/chemokine analysis present in the tissue lysate is used to produce a comprehensive biomarker. This data also include the micro RNA (miRNA) analysis and expression profiling (RNA-Seq) of immune cells present in the microenvironment of thyroid cancer to pinpoint the mechanism(s) involved in the onset and/or progression of the disease. This application further establishes the integration of the information derived from transcriptome/meta-analysis of the immune cell genome to the cellular profiling and cytokine/chemokine signals recorded from an FNA lysate.

### Example I

Risk of Developing Thyroid Cancer

Thyroid cancer is usually surrounded by a significant number of immune “reactive” cells.

Tumor associated lymphocytes and macrophages (TAL and TAM) are frequently described in pathology reports of patients operated for thyroid cancer. The nature of this lymphocytic reaction is not well understood. However, the fact that cancer can survive in this adverse immune microenvironment implies immune regulation.

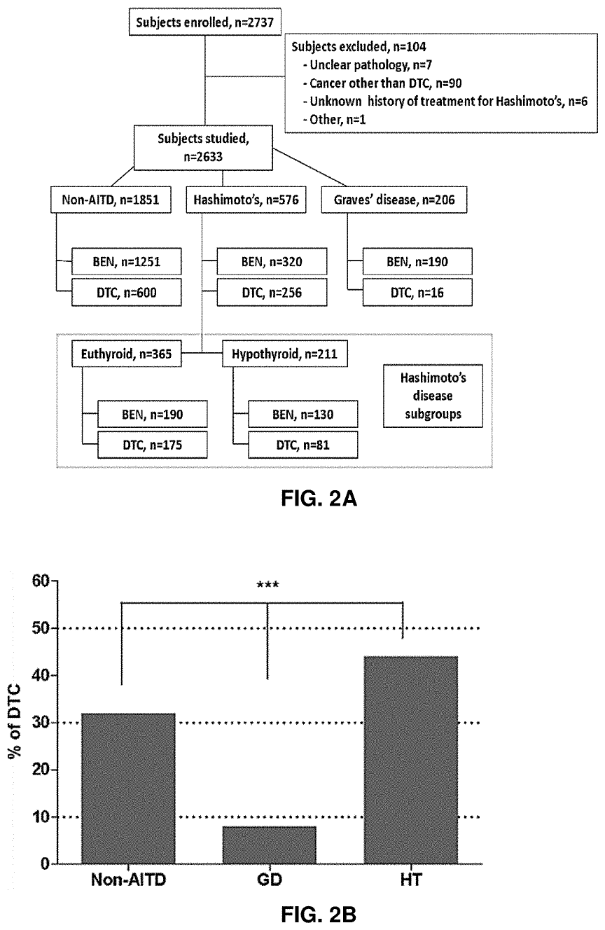

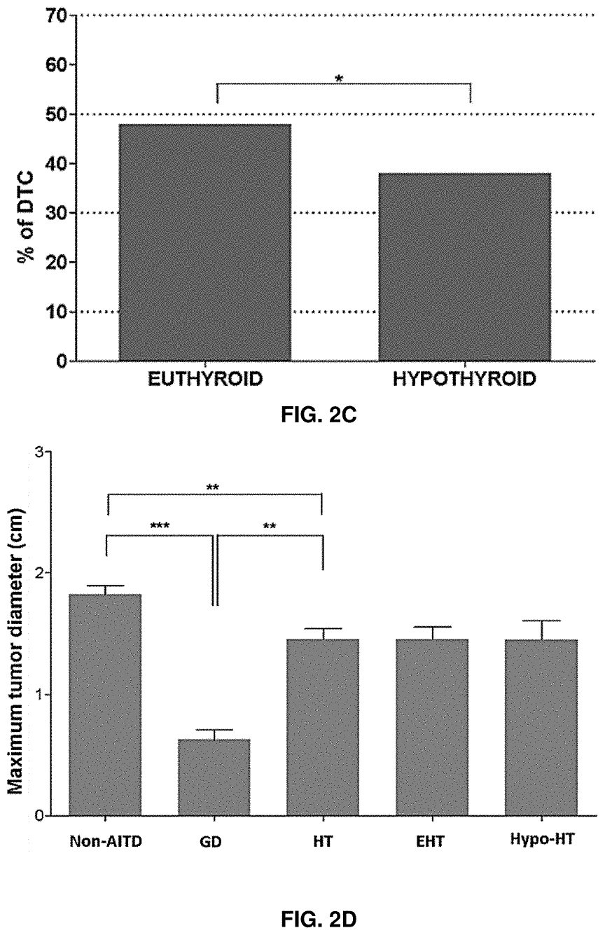

The risk of developing thyroid cancer is higher in patients with a silent form of autoimmune thyroid disease, Euthyroid Hashimoto Thyroiditis (EHT) as compared to GD and patient with non-autoimmune thyroid diseases (non-AITD) (FIG. 2B). The risk is especially pronounced in patients with functional thyroids and undetectable/low titers of thyroid peroxidase antibodies (TPO), while diminished in patients with full thyroid failure and high TPO antibody titers.

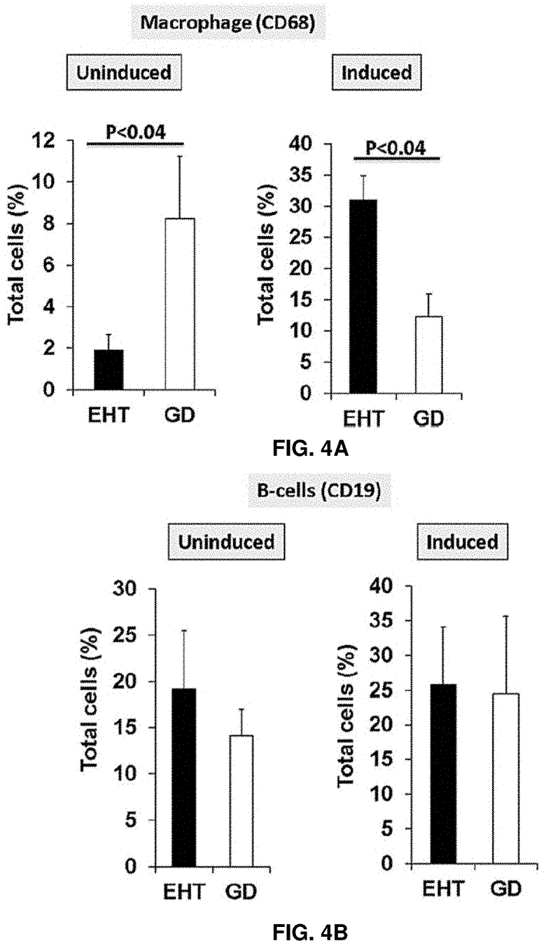

GD and EHT were investigated. Both are thyroid autoimmune diseases having no major differences in the B-cell (CD19) population (FIG. 4B). However, the risk for developing thyroid cancer in GD is less than 8%, whereas in EHT it is 48%. The cellular and molecular mechanisms behind this risk disparity were evaluated.

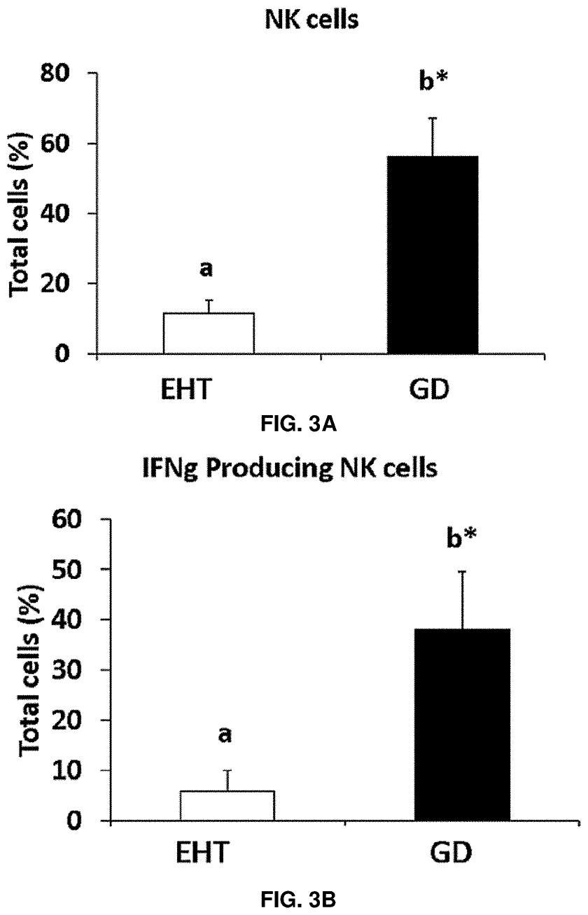

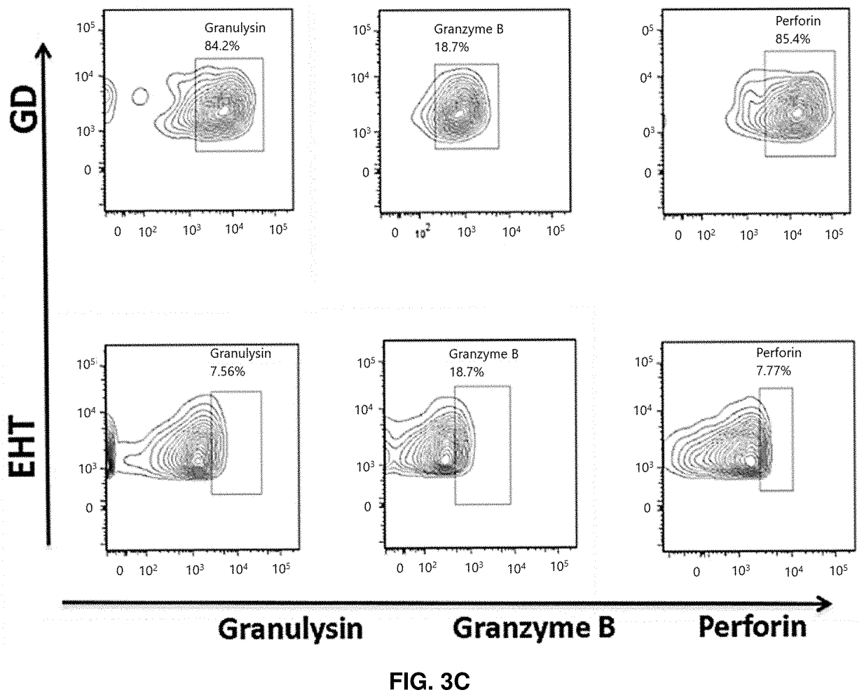

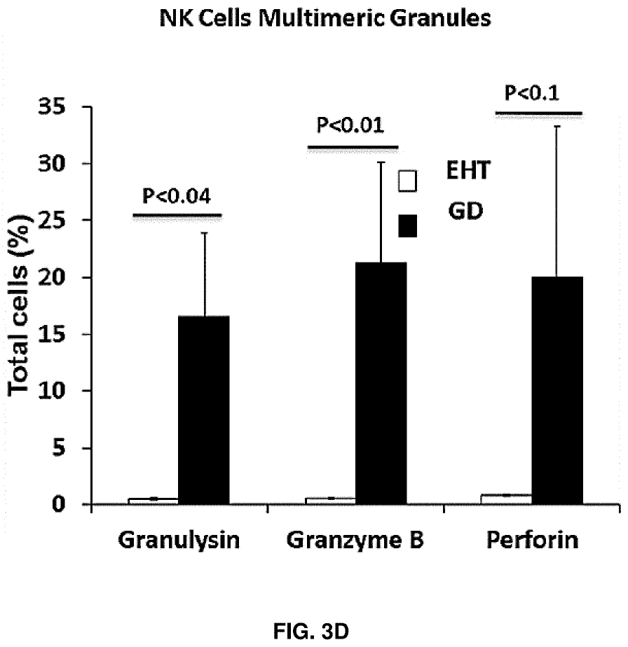

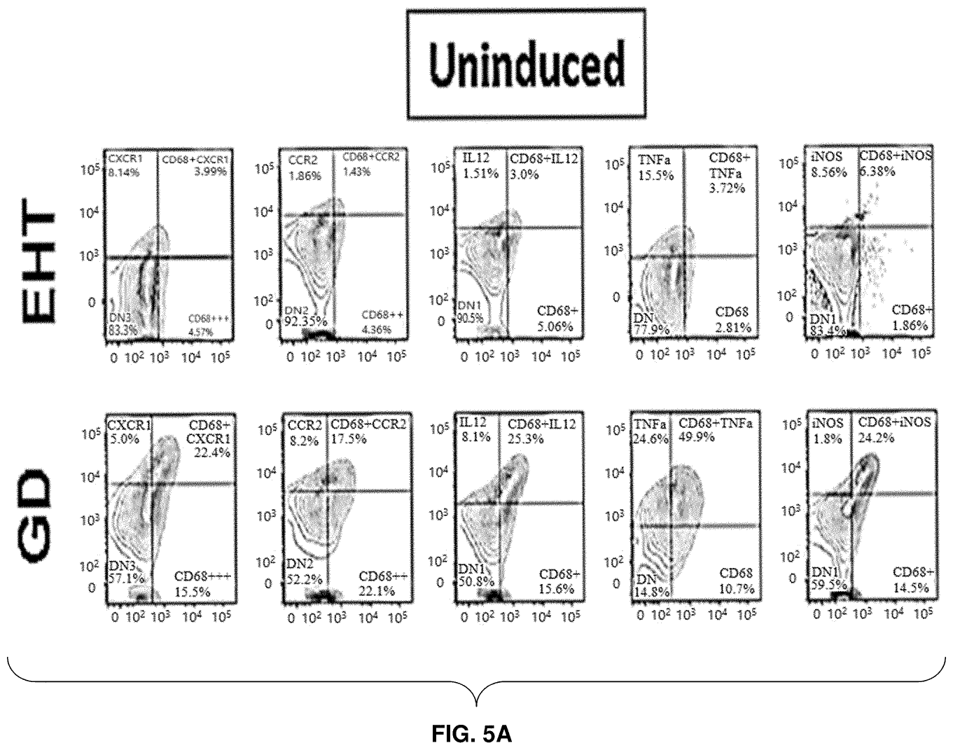

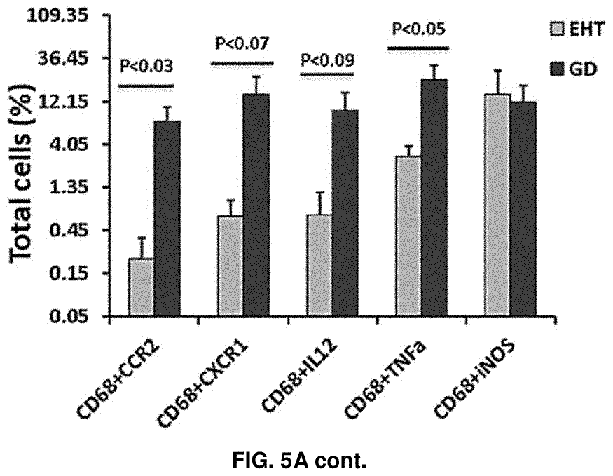

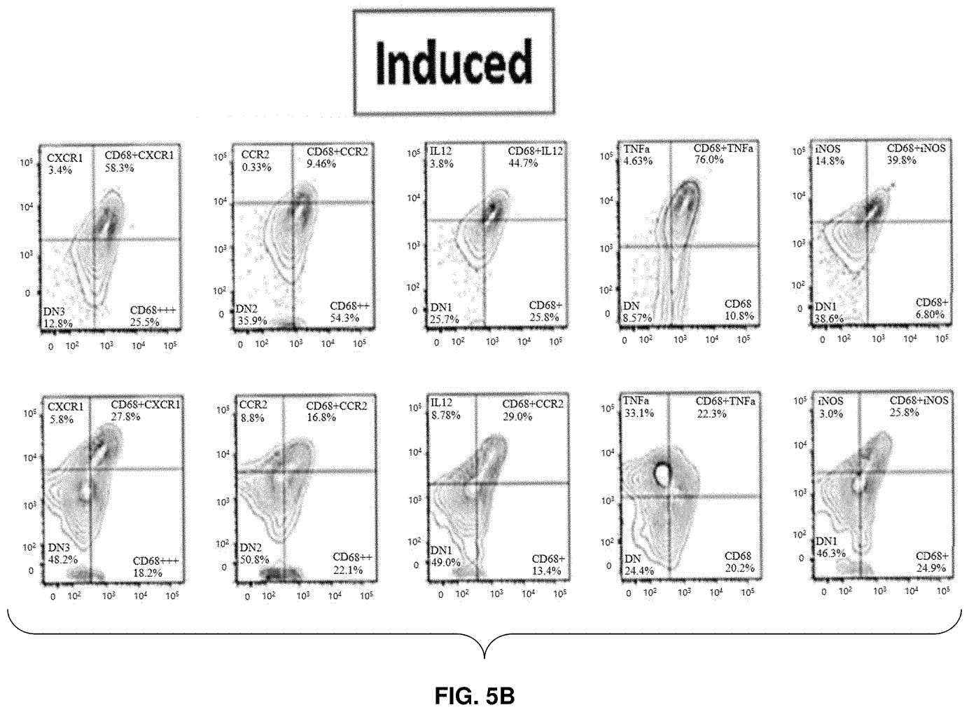

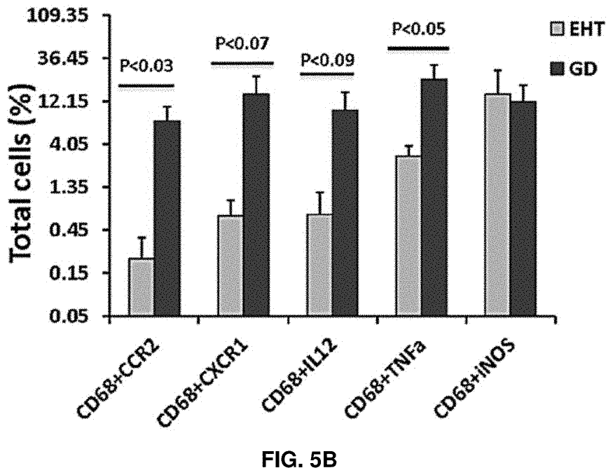

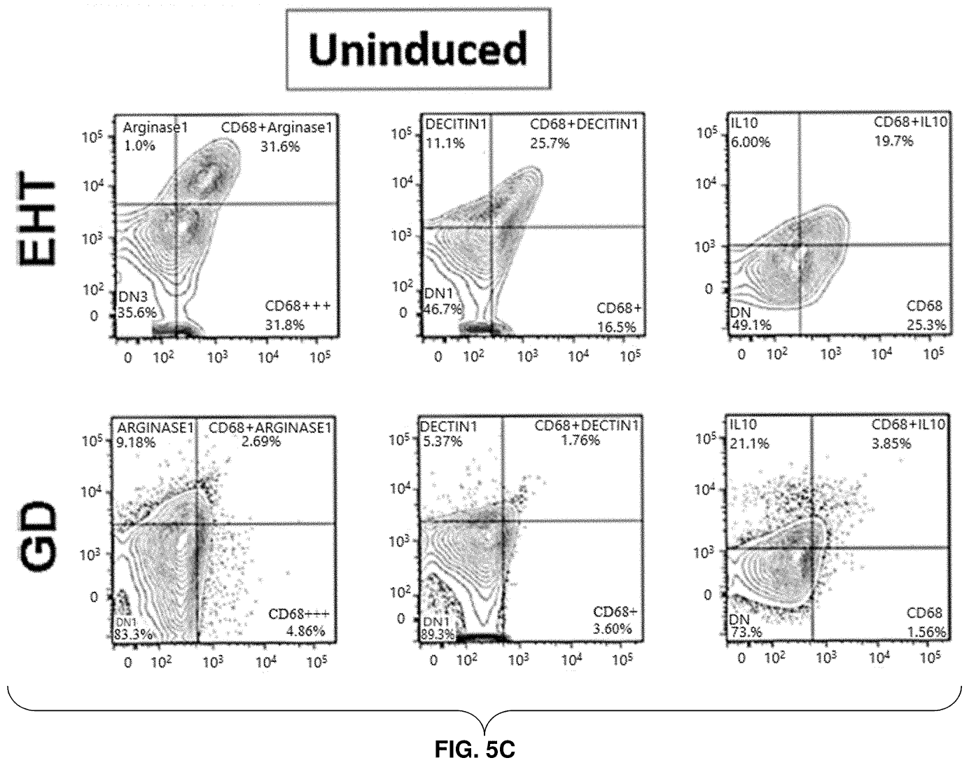

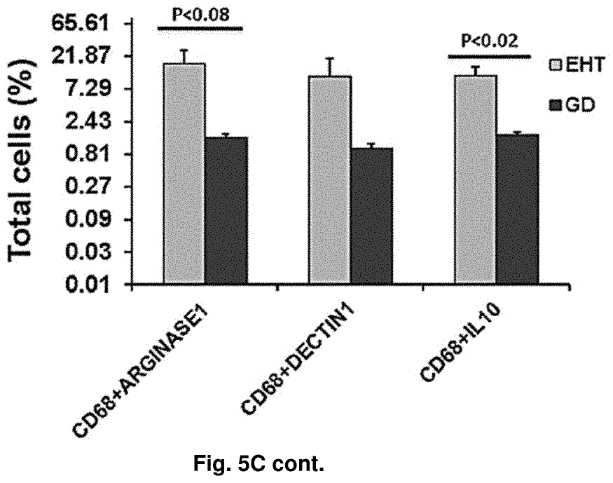

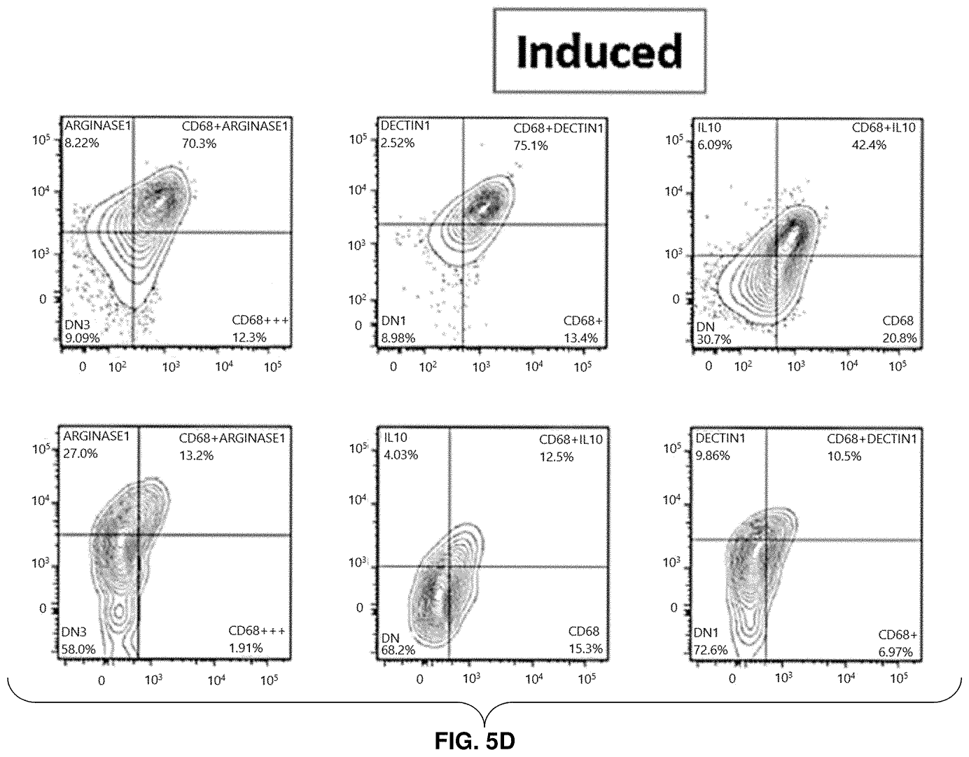

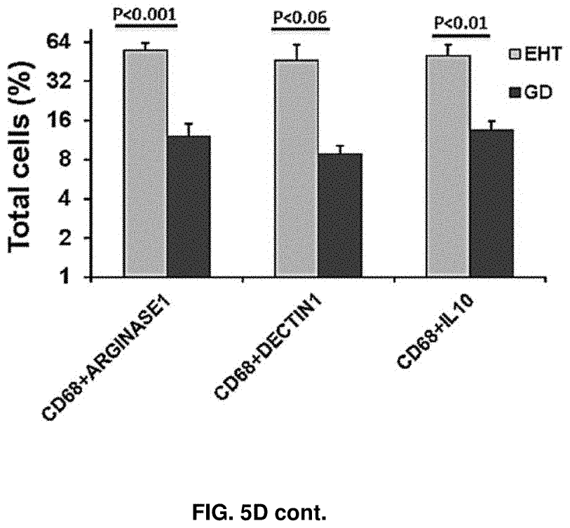

Data from 2633 consecutive patients with GD, HT, EHT, and non-Autoimmune Thyroid Disease (non-AITD) was analyzed for the presence of Differentiated Thyroid Cancer (DTC). The microenvironment and cellular mechanism of protection from DTC in GD/EHT were further investigated by ex-vivo aspirating infiltrates from thyroid samples. The results showed that DTC was less frequent/aggressive in GD as compared to EHT or non-AITD (FIG. 2). Intra-thyroidal immune-cell profiling revealed differential Natural Killer (NK) cell activity and macrophage polarization in the settings of GD versus EHT (FIGS. 3A-3B). In GD, NK cells were activated, and macrophages showed M1-like phenotype, whereas in EHT, NK-cells were less active and macrophages displayed M2-like phenotype (FIGS. 5A and 5C).

Overall, this example illustrates that the immune cellular phenotype dictates the fate of thyroid cancer progression. NK cell and M1 dominant microenvironments have a protective role, whereas depleted/low NK cells and M2 macrophages dominance promote the progression of cancer.

Tumor Associated DN T Cells

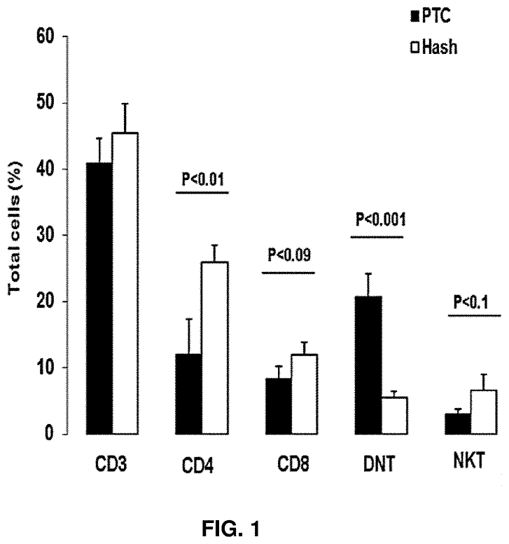

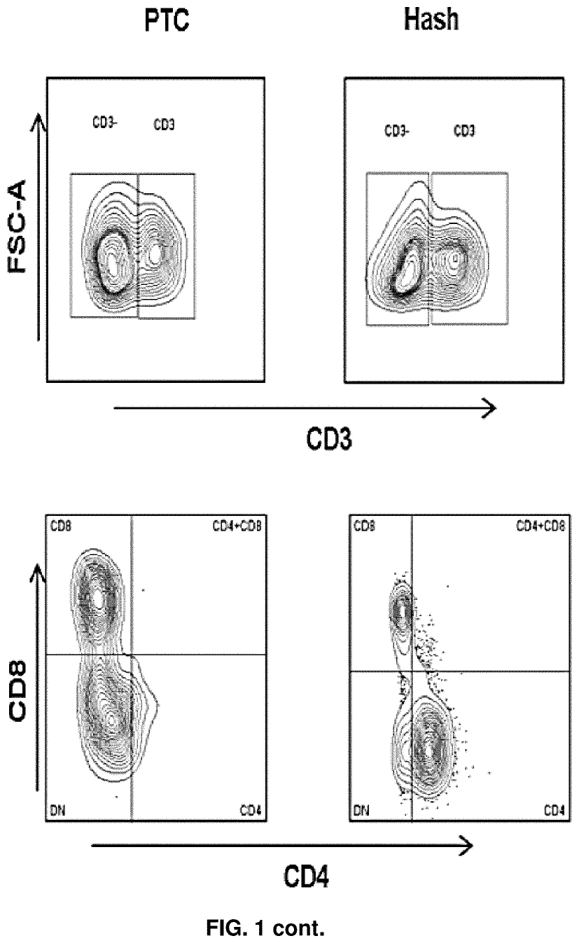

Intra-thyroidal lymphocytes accompanying thyroid cancer were observed to be reactive to the presence of the tumor, and these lymphocytes are different from lymphocytes found in autoimmune thyroid disease. The lymphocytic microenvironment of human postsurgical thyroid specimens was systematically characterized. Certain T cells not previously described in the setting of thyroid cancer, namely, CD3+ve CD4−ve CD8−ve (double negative, DN) T cells, were significantly more abundant in lymphocytic infiltrates of thyroid cancer and only traceable in controls (FIG. 1).

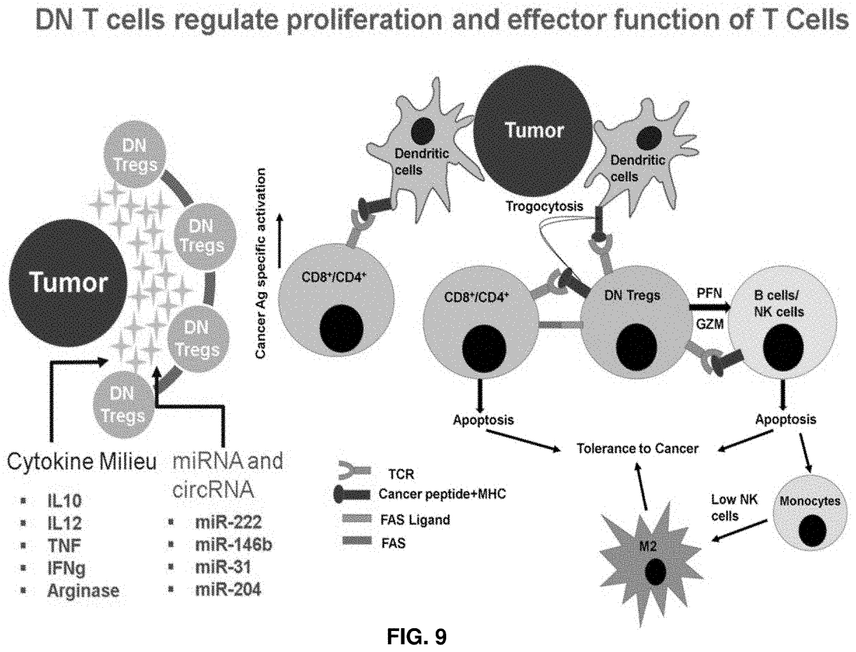

FIG. 9 is an illustration showing DN T cells regulate proliferation and effector function of T cells. Fas-FasL leads to activation-induced cell death, a form of apoptosis induced by repeated TCR stimulation, responsible for the peripheral deletion of activated T cells. Naturally Fas-FasL resistant DN T cells also induce apoptosis to NK cells. Absence of, or a low count of, active NK cells, plus macrophage plasticity, allows macrophage subtype M0 to differentiate to the M2 phenotype (precancerous/cancerous).

DN T cells were 20 times more abundant than classic Tregs (CD3+CD4+CD25+FoxP3+) in thyroid cancer samples, whereas CD4+ve and CD8+ve T cell counts were significantly lower in thyroid cancer samples. Thus, DN T cells downregulate the proliferation, and cytokine production of activated effector T cells present in the tumor microenvironment, contribute to tumor tolerance and active avoidance of tumor immunity (FIG. 9.) Therefore, tumor-associated DN T cells have an immunomodulatory role in papillary thyroid cancer (PTC) for example.

Using tissue extracted from patients undergoing a thyroid removal, the DN T cell frequency (i.e., the proportion of DN T cells in lymphocytes) was used to predict thyroid cancer, with the predictions being validated by post-operative diagnosis. The tissue samples analyzed were extracted via FNA from areas within and adjacent to the thyroid nodule of concern. The results are summarized in Tables 1 and 2 below.

TABLE 1
Results of first study

| | | Post- | | Levothyroxine | | Post- | |
|---|---|---|---|---|---|---|---|
| Study | Pre-operative | profile | | replacement | Risk for | operative | DNT |
| ID | diagnosis | diagnosis | Status | dose | cancer | diagnosis | Freq. |
| A | Goiter | HASH | Euthyroid − | — | “Low risk” | EUHASH | 10.41 |
| | | | TPO + | | | | |
| B | Dysphagia | HASH | Hypothyroid − | 1.22 mcg/Kg | Low risk | HASH | 4.09 |
| | | | TPO + | | | | |
| C | Dyspnea, OSA | HASH, | Hypothyroid − | 1.06 mcg/Kg | Low risk | HASH | 5.3 |
| | | FA | TPO + | | | | |
| D | Dysphagia, | HASH | Hypothyroid − | 2.87 mcg/Kg | Low risk | HASH | 3.342 |
| | SOB | | TPO + | | | | |
| E | FLUS | HASH | Euthyroid − | | Very high | EUHASH | 23.87 |
| | | | TPO − | | risk | | |
| F | PTC | PTC- | Hypothyroid − | 1.7 mcg/Kg | High risk | PTC- | 19.8 |
| | | HASH | TPO + | | | HASH | |
| G | PTC | PTC- | Euthyroid − | | Very high | PTC- | 28.7 |
| | | HASH | TPO − | | risk | EUHASH | |
| H | PTC | PTC- | Hypothyroid − | 0.67 mcg/Kg | Intermediate | PTC- | 33.47 |
| | | HASH | TPO + | | risk | EUHASH | |
| I | Compressive | HASH | Hypothyroid − | 1.07 mcg/Kg | Medium- | EUHASH | 13.7 |
| | symptoms | | TPO − | | high risk | | |
| J | FLUS | HASH | Hypothyroid − | 0.68 mcg/Kg | Low risk | HASH | 6.34 |
| | | | TPO + | | | | |
| K | FLUS | HASH | Hypothyroid − | 0.88 mcg/Kg | Low risk | HASH | 4.87 |
| | | | TPO + | | | | |

TABLE 2
Results of second study

| | Pre- | | | Levothyroxine | | Post- | |
|---|---|---|---|---|---|---|---|
| Study | operative | Post-profile | | replacement | Risk for | operative | DNT |
| ID | diagnosis | diagnosis | Status | dose | cancer | diagnosis | Freq./ste |
| 1 | MNG | MNG | TPO −ve | | Low risk | MNG | 2.65 |
| | | | | | | | 0.21 |
| 2 | Goiter | Hashimoto's- | Euthyroid | should get | should get | Hashimoto's- | 8.510 |
| | | Benign | HASH-high | hypothyroid | hypothyroid | Benign | |
| | | | TPO titer | soon | soon | | |
| | | | Benign due to | | | | 0.359 |
| | | | aggressive | | | | |
| | | | inflammation | | | | |
| 3 | Dysphagia | Hashimoto's- | Hypothyroid- | 1.22 mcg/Kg | Low risk | Hashimoto's- | 7.30 |
| | | Benign | High TPO titer | low risk | | Benign | |
| | | | | | | | 0.80 |
| 4 | Graves | Graves | TPO +ve | Low Risk | Low risk | Graves | 3.50 |
| | | | | | | | 0.30 |
| 5 | Dyspnea, | HASH | Hypothyroid- | 1.06 mcg/Kg- | Medium risk | HASH | 22.60 |
| | OSA | | High TPO titer | Medium risk | | | |
| | | | | | | | 0.32 |
| 6 | Graves | Graves | TPO +ve | Low risk | Low risk | Graves | 4.23 |
| | | | 1:100 | | | | |
| | | | | | | | 0.44 |
| 7 | Neoplasm | PTC | TPO +ve | Cancer | Cancer | PTC | 14.90 |
| | | | | | | | 1.60 |
| 8 | Dysphagia, | Hashimoto's | Hypothyroid- | 2.87 mcg/Kg | Zero risk | Hashimoto's | 7.50 |
| | SOB | benign | High TPO titer | | | benign | |
| | | | | | | | 0.00 |
| 9 | PTC | PTC | TPO −ve | High risk for | High risk for | PTC | 33.88 |
| | | | | cancer | cancer | | |
| | | | | | | | 2.61 |
| 10 | MTC | MTC | Not available | MTC | Cancer | MTC | 8.12 |
| | | | | | | | 0.44 |
| 11 | MTC | HCC | TPO +ve | | Cancer | HCC | 18.5 |
| | | | 1:100 | | | | |
| | | | | | | | 1.23 |
| 12 | FLUS | Hashimoto's | Euthyroid | High risk for | High risk for | Euthyroid | 9.387 |
| | | MNG | HASH-TPO | cancer | cancer | Hashimoto's | |
| | | | negative | | | MNG | |
| | | | | | | | 0.907 |
| 13 | PTC | PTC-HASH | Hypothyroid- | 1.7 mcg/Kg- | Cancer risk | PT- | 13.802 |
| | | | High TPO titer | | | HASH | |
| | | | | | | | 1.205 |
| 14 | Dysphagia | MNG | TPO −ve | | Low risk | MNG | 7.93 |
| | | | | | | | 0.91 |
| 15 | PTC | HASH-PTC | Euthyroid | High risk for | High risk for | EUHASH- | 24.54 |
| | | | HASH-low | cancer | cancer | PTC | |
| | | | TPO titer | | | | |
| | | | | | | | 0.74 |
| 16 | PTC | HASH-PTC | Hypothyroid- | 0.67 mcg/Kg- | High risk for | HASH-PTC | 25.000 |
| | | | High TPO titer | High risk | cancer | | |
| | | | | | | | 2.796 |
| 17 | PTC | PTC | Hypothyroid | 0.3 mcg/Kg | Cancer | PTC | 11.274 |
| | | | Low TPO titer | | | | |
| | | | | | | | 1.195 |
| 18 | Compressive | HASH | Hypothyroid- | 1.07 mcg/Kg | High risk for | HASH | 30.475 |
| | symptoms | | Low TPO titer | | cancer | | |
| | | | | | | | 0.637 |
| 19 | Cellular | MNG | TPO −ve | | Low risk for | MNG | 3.355 |
| | nodule | | | | cancer | | |
| | | | | | | | 0.820 |
| 20 | FLUS | HASH s/p | Hypothyroid- | Low risk for | Low risk for | HASH | 5.788 |
| | | radiation | High TPO titer | cancer | cancer | | |
| | | | | | | | 0.607 |
| 21 | Benign | Benign | Not available | Low risk | Low risk for | Benign | 1.754 |
| | | | | | cancer | | |
| | | | | | | | 0.116 |
| 22 | FLUS | HASH | Hypothyroid- | 0.88 mcg/Kg | Low risk for | HASH | 9.140 |
| | | | TPO | Low risk for | cancer | | |
| | | | | cancer | | | |
| | | | | | | | 1.294 |
| 23 | Goiter | MNG | TPO −ve | | Low risk for | MNG | 1.460 |
| | | | | | cancer | | |
| | | | | | | | 0.123 |
| 24 | PTC | PTC | TPO −ve | High risk for | High risk for | PTC | 33.125 |
| | | | | cancer | cancer | | |
| | | | | | | | 1.657 |

Notably, in most cases where the DN T cell frequency was 15 or higher, the thyroid nodule was or became cancerous (as indicated by “PTC”, which stands for papillary thyroid cancer). Further, in most cases where the DN T cell frequency was 5 or lower, the thyroid nodule was not, and did not become, cancerous.

Methodology

Direct Detection of Inflammatory Mediators by Using the BD Multiplexed Bead-Based Immunoassay in Intra-Thyroidal FNA Aspirates.

Intra-thyroidal FNA aspirates is centrifuged for separating cells from supernatants. The concentrations of inflammatory signatures/chemokines/cytokines in thyroid FNA supernatant is measured using a BD, custom CBA Flex Set, enhanced sensitivity for human inflammatory cytokine Eotaxin, IFNα, IFNγ, IL-1α, IL-1β, IL-2, IL-3, IL-4, IL-5, IL-6, IL-7, IL-8, IL-10, IL-12p70, IL-12p40, IL-13, IL-15, IL-17A, IL-33, IP-10, MCP-1, MIP-1a, MIP-1b, TNFα, TNFβ, TGFβ1, TGFβ2, and TGFβ3, according to the manufacturer's protocol. Fifty microliters of FNA supernatant/tissue lysate or standard is incubated with 50 μL of capture beads for 1 h at room temperature, then mixed with 50 μL of phycoerythrobilin (PE)-labeled detection antibodies and incubated for 2 h at room temperature to form a sandwich complex. Following incubation, 1 mL of washing buffer (BD Biosciences Pharmingen, United States) is added to each tube, and the mean fluorescence intensity is detected using flow cytometry (LSRII, BD Co, United States). Data is analyzed using BD FACSArray analysis software.

Intra-Thyroidal FNA Aspirated Leukocyte Profiling

The FNA is aspirated and centrifuged to collect infiltrating leukocytes. The red blood cells (RBC) in the aspirates is lysed by a brief hypotonic shock and the rest of the cells is suspended in RPMI-1640 media containing 10% Fetal Calf Serum (FCS) at 40° C. The cells is stained for surface staining with fluorochrome-conjugated antibodies against human (CD45, CD3, CD192/CCR2, CX3CR1, CD14, CD16, CD19, CD56, CD335/NKp46 (BD Biosciences, San Jose, Calif.), or isotype controls in serum-containing media.

Briefly, the cells is incubated with antibodies for 20 minutes on ice for surface staining, washed, and fixed in 1% paraformaldehyde. Aliquots of cells are permeabilized with cytofix/cytoperm solution (BD Biosciences), and intracellular staining is performed using fluorochrome conjugated antibodies against human [CD68, TNFa, Interleukin (IL)-10, IL-12/23p40, Perforin, Granzyme B, Granulysin, IFNg, Arginase 1/ARG1, Dectin□1/CLEC7A, iNOS (BD Biosciences, San Jose, Calif.)]. Cells can also be stained with Hoechst 33342 (10 μg/ml for 2 hr, Hoechst fluorescence, 350 nm excitation/450 nm emission, linear scale) used to gate live cells containing 2n-4n cellular DNA.

RNA-Seq

The FNA is aspirated and centrifuged to collect infiltrating leukocytes. The RNA-Seq method most commonly used is the next generation sequencing technique. The fresh obtained centrifuged tissue sample is snap frozen in liquid nitrogen and stored at −80° C. for total RNA extraction. In all RNA-Seq approaches, RNA is converted into cDNA using the enzyme reverse transcriptase. After total RNA isolation from tissues, RNA is either directly processed into cDNA to obtain a full catalog of RNAs (total RNA-Seq) or selective enrichment of RNAs of interest is done before synthesizing cDNA. For mRNA enrichment, one example methodology is called mRNA-Seq. The rRNA removal is performed either using the poly(A) enrichment or ribo-depletion RNA approaches. The rRNA (ribo) depletion, achieved by hybridizing total RNA to bead bound rRNAs, is commonly used in RNA-seq template preparation, particularly when working with formalin-fixed clinical samples. After the selective enrichment, the cDNA or RNA is fragmented into short sequences of 200-500 nt, which can easily be sequenced. The sequencing of these fragments results in short reads that are aligned with the reference genome and further analyzed.

For circRNA-Seq, the total RNA isolated from tissues is subjected to rRNA depletion and RNase R treatment. RNase R can degrade all the linear RNAs present in total isolated RNA. The remaining RNAs are used for library construction followed by sequencing of these libraries using Illumina Hiseq 2500 analyzer. This is followed by trimming the adapter sequences and filtering low quality reads. The final generated clean reads with high quality is subjected to optimized pipeline to identify circRNAs.

For exploring miRNA expression, total RNA is isolated from cultured cells with TRIzol (Invitrogen, USA) according to the manufacturer's instructions. MiRNA is polyadenylated and reverse transcribed using poly(A) polymerase and MMLV reverse transcriptase (Clontech, USA).

Bioinformatics analysis is performed using different softwares, including KEGG pathway analysis. Datasets are also analyzed with ToppGene Suite25 for gene list enrichment analysis and candidate gene prioritization. ToppFun can also be used for to generated gene list functional enrichment analysis, gene ontology (GO) molecular function, GO terms, pathways, protein-protein interactions, protein functional domains, transcription factorbinding sites, microRNAs, gene tissue expressions, and literature searches.

### Example II

Nature of Coexisting Thyroid Autoimmune Disease Determines Success or Failure of Tumor Immunity in Thyroid Cancer

Thyroid cancer and thyroid autoimmunity are considered opposite extremes of immune-responses. However, it has been shown that thyroid cancer coexists with autoimmune thyroid diseases such as Hashimoto Thyroiditis (HT) and Graves disease (GD). In accordance with the present disclosure, the risk of developing thyroid cancer is higher in patients with a silent form of autoimmune thyroid disease-Euthyroid Hashimoto Thyroiditis-(EHT).

In this example, data from 2633 consecutive patients with GD, HT, EHT, and non-autoimmune thyroid disease (non-AITD) were analyzed for the presence of Differentiated Thyroid Cancer (DTC) (FIGS. 2A-2D). The microenvironment and cellular mechanism of protection from DTC in GD/EHT was further investigated by ex-vivo aspirating infiltrates from thyroid samples.

Further, the in-vivo microenvironment was re-constituted in vitro to mimic an in-vivo context. NK cells were isolated and macrophages were differentiated into M1 and M2 phenotype from healthy human peripheral blood monocytes.

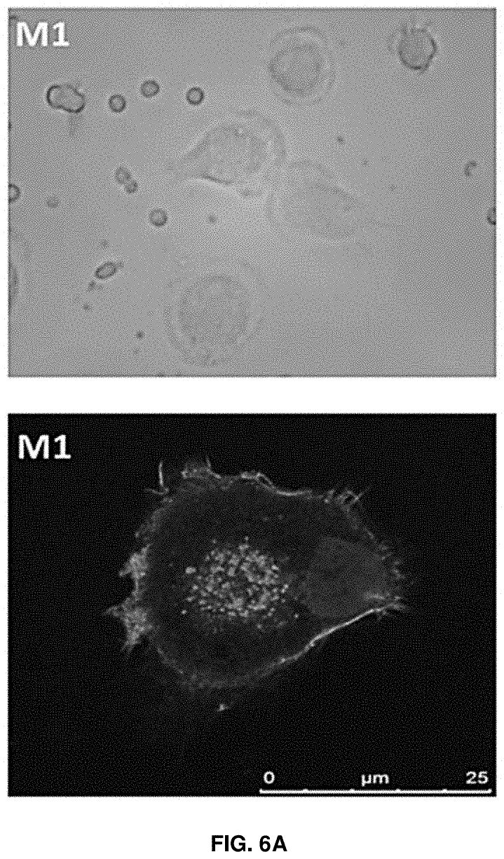

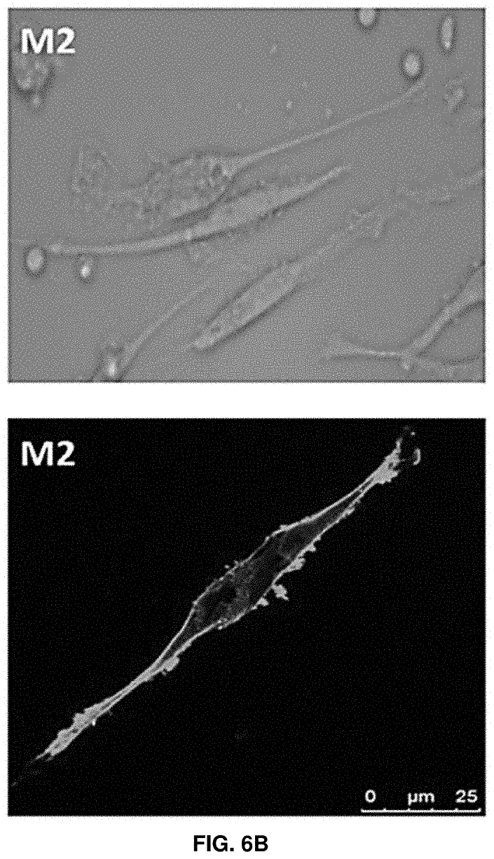

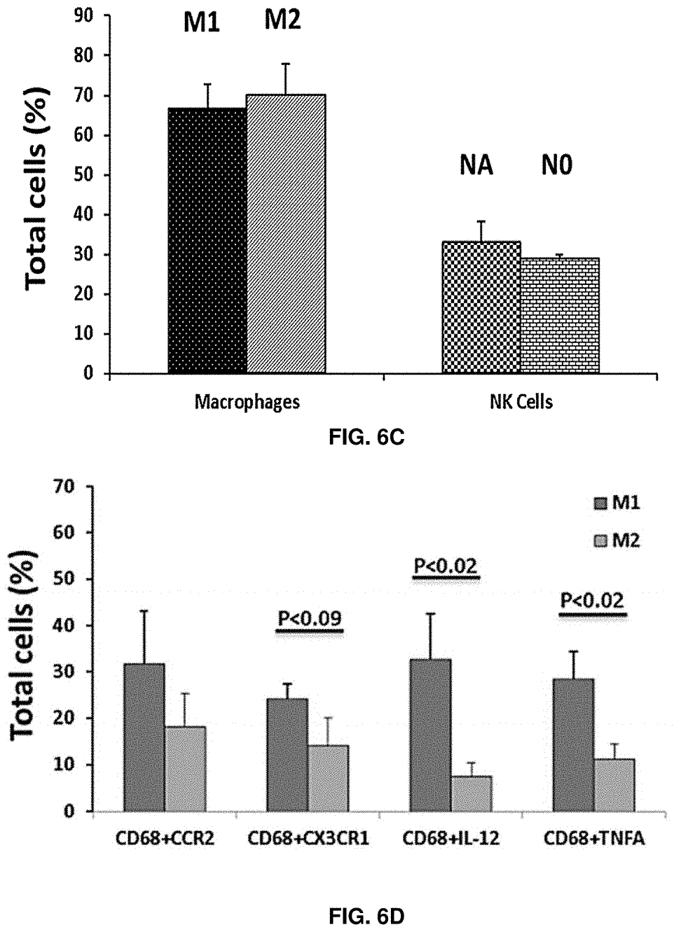

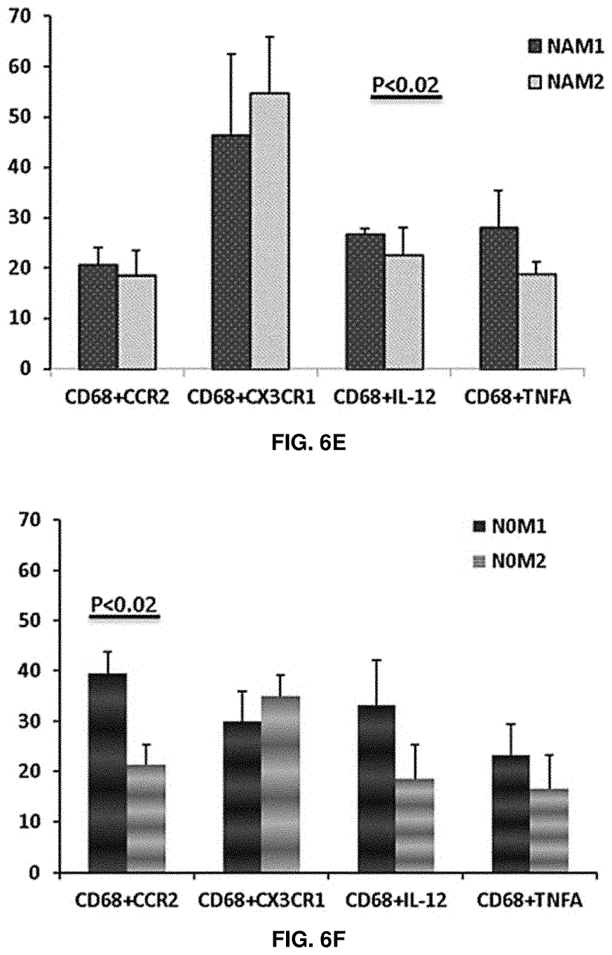

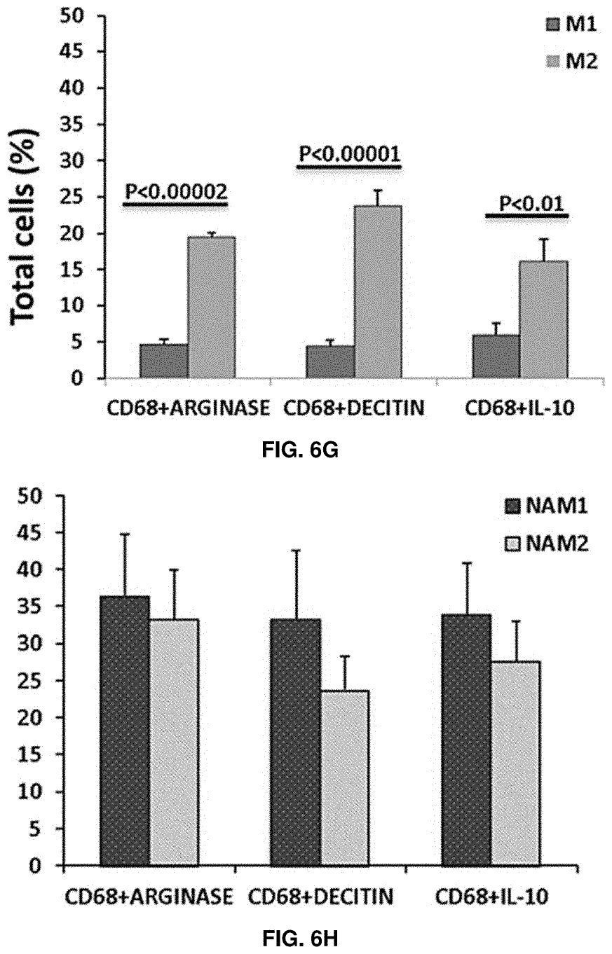

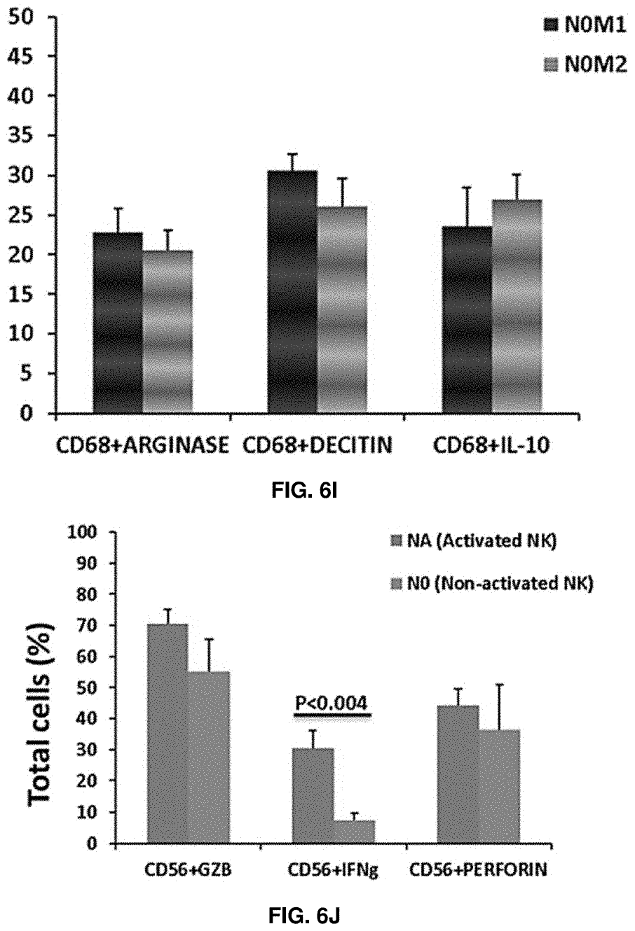

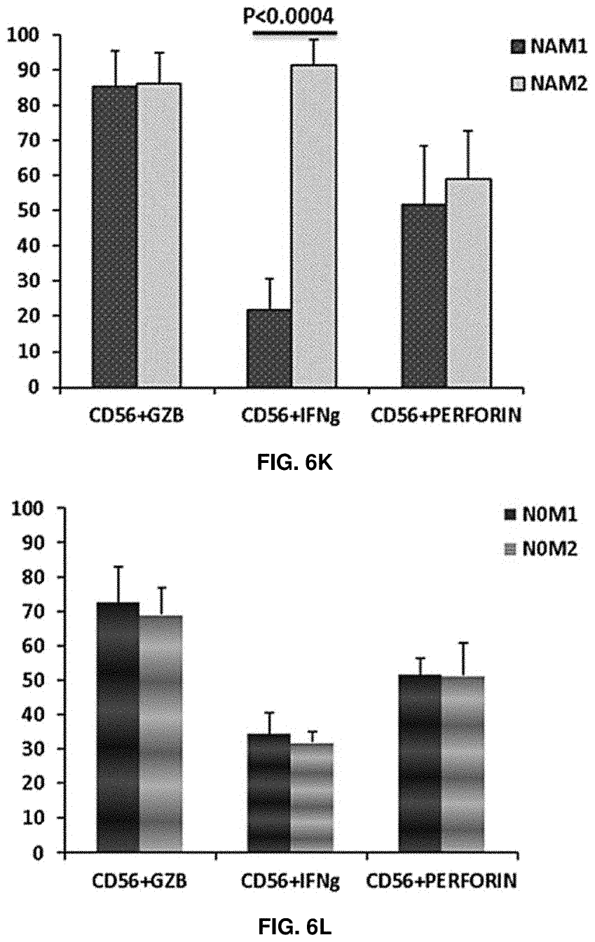

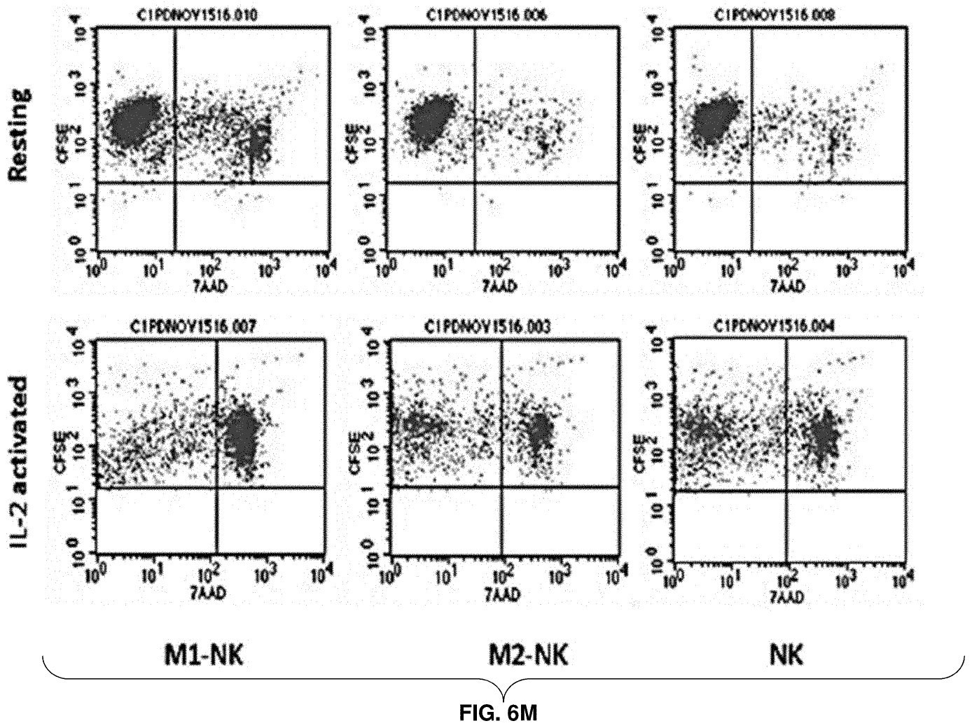

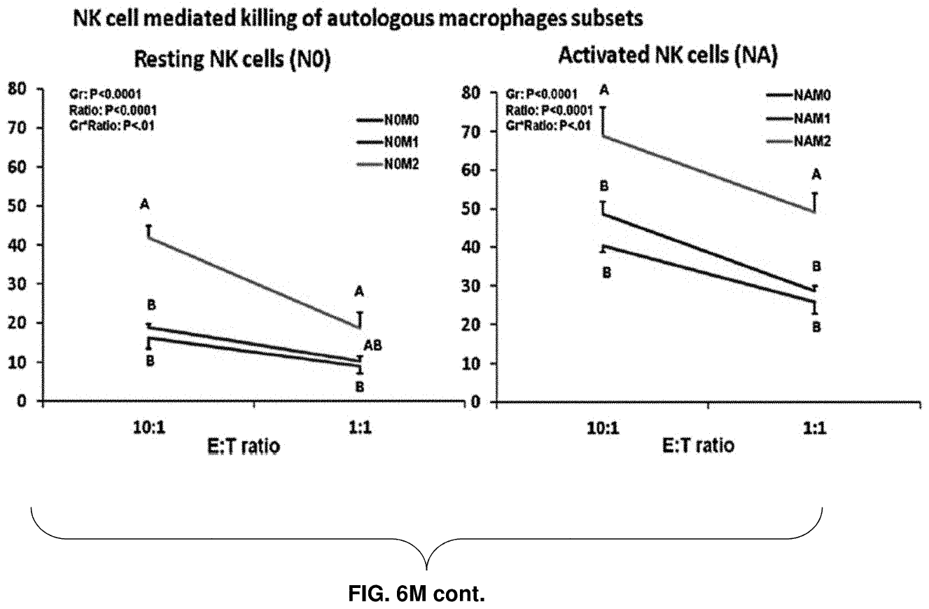

The results showed that DTC was less frequent/aggressive in GD as compared to EHT or non-AITD. Intra-thyroidal immune-cell profiling revealed differential natural killer (NK) cell activity and macrophage polarization in the settings of GD versus EHT. In GD, NK-cells were activated, and macrophages showed M1-like phenotype whereas in EHT, NK-cells were less active and macrophages displayed M2-like phenotype (FIGS. 5A and 5C). Furthermore, in vitro co-cultures of NK-cells with differentiated macrophage subsets revealed that the presence of activated NK (NA) cells favors M1 macrophages, boosts macrophage action, and amplifies the innate defense mechanisms. Moreover, co-culture of M2 macrophages with NA increases the cytotoxicity of NK-cells and favors a pro-inflammatory microenvironment that reverts the anti-inflammatory M2 towards pro-inflammatory M1 (FIGS. 6A-6L).

Surveillance innate immune-cells like natural killer (NK) cells and macrophages are complementary to each other in their actions. It was observed here that activated NK-cells in the background of the thyroid autoimmune disease, GD, drive macrophage differentiation to the M1/killer phenotype which, in turn, is cytotoxic to cancer cells and down regulates the M2/repair phenotype. Understanding the molecular basis of macrophage-NK cell interface in Thyroid Cancer, ETH, and GD opens the door for certain immune therapeutic intervention. Macrophages/innate immunity is modulated from M2 to M1 phenotype to help treat thyroid cancer as naturally done by GD.

Moreover, these findings demonstrated that DN T cells regulate proliferation and effector function of T cells. Fas-FasL leads to activation-induction of cell death, a form of apoptosis induced by repeated TCR stimulation, responsible for the peripheral deletion of activated T cells. Naturally Fas-FasL resistant DN T cells also induce apoptosis to NK cells. Absence of, or a low count of, active NK cells, macrophage plasticity, allows macrophage subtype M0 to differentiate to the M2 phenotype (precancerous/cancerous).

Description

Antitumor immune responses are often detected in human cancers, but in many cases, do not control tumor progression and/or may favor tumor growth. In organ-specific autoimmune diseases, immune responses are lethal to the target organ. Memory immune cells persist for life and facilitate chronic target destruction even under immunosuppressive conditions.

Thyroid cancer and thyroid autoimmunity appear to be situated at opposite extremes of the immune response spectrum. In thyroid cancer the immune response seems tolerant, allowing for tumor growth. In thyroid autoimmunity the immune response is destructive, usually leading to thyroid failure.

While known to immunologists for decades, it has surfaced in recent years that more effective immune-therapy of cancer is associated with autoimmunity. The interplay between autoimmunity and cancer is now taking center stage after the introduction of “immune check-point inhibitors” for cancer treatment. Favorable outcomes with these cancer immunotherapeutic drugs are now clearly associated with endocrine immune-related adverse effects like autoimmune thyroid disease and type 1 diabetes.

In cancer development, progressive accumulation of genetic abnormalities renders cells malignant. The immune system seems to be allowing or even promoting cancer progression for some tumors. While immune regulation in cancer seems to uphold development and progression, immune dysregulation in autoimmunity leads to tissue destruction and target organ elimination.

Thyroid cancer is usually surrounded by a significant number of immune “reactive” cells. Tumor associated leucocytes and macrophages (TAL and TAM) are frequently described in pathology reports of patients operated for thyroid cancer. Macrophages play an important role in the progression/regression of cancer. Macrophage phenotype which stimulate tumor growth is M2/repair type whereas macrophages which inhibit/slow the tumor growth are M1/kill-type. The nature of this leucocytic reaction is not well understood. Evidently, the fact that cancer can survive in this adverse immune microenvironment speaks for immune regulation.

The risk of developing thyroid cancer is higher in patients with a silent form of autoimmune thyroid disease-Euthyroid Hashimoto Thyroiditis. The risk is especially pronounced in patients with functional thyroids and undetectable/low titers of thyroid peroxidase antibodies (TPO), while diminished in patients with full thyroid failure and high TPO antibody titers.

Some previous studies have suggested that thyroid cancer coexisting with another form of autoimmune thyroid disease (Graves disease) might be more frequent and aggressive as compared to that found in patients without Graves. However, the clinical observations described in these examples argue against this.

In the present example, the roles of humoral and cellular autoimmunity in the development of thyroid cancer and the likelihood of less aggressive behavior in the setting of Graves disease were investigated, combining epidemiological and laboratory data.

Materials and Methods

Thyroid Subjects

The Thyroid Multidisciplinary Clinic is a large referral site for thyroid diseases. Patients referred for thyroid surgery include those with cytology positive or suspicious for malignancy on fine needle aspiration (FNA), and those with nodular goiter associated with compressive symptoms (such as dysphagia, shortness of breath, or hoarseness). Random patients undergoing thyroid surgery had their thyroids ex-vivo aspirated in the operating room (FIG. 2A). A tissue sample from each of those patients was also snapped frozen in liquid nitrogen and stored for further analysis. Post-operative histology confirmed the presence of multinodular goiter, GD, or HT, with or without DTC. The subjects with Hashimoto's were split by histology into two subgroups: those with established hypothyroidism (abnormally high TSH and low free T4 preoperatively, treated with levothyroxine), and those with normal thyroid function (euthyroid Hashimoto thyroiditis) (FIG. 2A). Patients with preoperative hypothyroidism which was a result of previous thyroid surgery or radioactive iodine treatment were excluded. In patients with Graves' histology, the diagnosis was confirmed prior to surgery based on the presence of clinical hyperthyroidism with abnormally low TSH and high free T4, along with either exophthalmos, homogeneously enhanced Tc-99m uptake, or elevated titers of TSH receptor antibodies. Post operatively, established pathological characteristics for the diagnosis of multinodular goiter, thyroid cancer, Graves', and Hashimoto's were followed by academic pathologists. Patients were invited to participate and signed consent pre-operatively.

For all patients, the following data was collected: gender, age, and TSH, thyroid autoantibodies titers [TPO-Abs, Thyroglobulin antibodies (Tg-Abs), TSH receptor antibodies (TR-Abs), TSH receptor stimulating immunoglobulins (TSI)] when available, and the surgical pathology report. TSH concentrations and thyroid autoantibodies titers were measured by chemiluminescent immunoassays.

Patients on levothyroxine (LT4)-suppressive therapy (when used to prevent growth of a goiter or thyroid nodules), patients with prior exposure to radioactive iodine, prior thyroid surgery, or patients with incomplete records were excluded. No GD patients were treated with SSKI prior to surgery. Some patients received anti-thyroidal drugs prior to surgery but they demonstrated no statistical differences on the leukocyte infiltrates.

Intra-Thyroidal Leukocyte Profiling

The resected thyroid glands were aspirated ex-vivo to collect infiltrating leukocytes. The red blood cells (RBC) in the aspirates were lysed by a brief hypotonic shock and the rest of the cells were suspended in RPMI-1640 media containing 10% Fetal Calf Serum (FCS) at 40° C. The cells were stained for surface staining with fluorochrome-conjugated antibodies against human or isotype controls in serum-containing media as described earlier.

Briefly, the cells were incubated with fluorochrome-conjugated antibodies for 20 min on ice for surface staining, and then washed and fixed in 1% paraformaldehyde. Aliquots of cells were permeabilized with cytofix/cytoperm solution (BD Biosciences) and intracellular staining was performed using fluorochrome-conjugated antibodies against human. Cells were also stained with Hoechst 33342 (10 μg/ml for 2 h, Hoechst fluorescence, 350 nm excitation/450 nm emission, linear scale) used to gate live cells containing 2n-4n cellular DNA.

In Vitro Stimulation/Induction

Aliquots of thyroid infiltrating leukocytes/thyroid cells were stimulated (induced) in vitro with lipopolysaccharide (LPS) (100 ng/ml) and incubated in RPMI-1640 media with 10% FCS for 54 h at 37° C. in a 5% CO, atmosphere. In the last 6 h of induction, cells were treated with Golgi plug brefeldin A (1 μg/ml), (Sigma-Aldrich, St, Louis, Mo.) for intracellular cytokines analysis. The un-stimulated cells served as controls.

NK/Macrophage Co-Culture

NK cells were isolated from healthy human peripheral blood using human NK isolation kit (Miltenyi Biotech, USA) as described by the manufacturers. Prior to co-culture, NK cells were plated in RPMI-1640 media plus 10% FCS with or without human IL-2 (50 ng/ml, Life Technologies Corporation, Grand Island, N.Y.) at 37° C. for 16 h.

Macrophage subsets were differentiated from healthy human monocytes using M1 and M2 macrophage generation medium (PromoCell, United States) as described by the manufacturer. M0 subsets represent macrophages differentiated from monocytes without adding M1/M2 polarizing cytokines to the media. Prior to co-culture, the macrophage subsets were washed three times with 10% FCS containing medium.

Resting (N0) or IL-2 activated NK (NA) cells were co-cultured with autologous macrophage subsets (M0, M1, and M2) at 1:1 ratio in RPMI-1640 media with 10% FCS for 72 h. Macrophages were analyzed for the expression of M1 or M2 lineage specific markers by flow-cytometry. NK cells were analyzed for NK cell marker (CD56) and intracellular cytokine expression (Granulysin, Granzyme B, Perforin, and IFNg) also by flow cytometry.

Cytotoxicity Assay

Natural killer (N0 or NA) cells were co-cultured with K562 cells (ATCC, cat #CCL-243) or macrophage subsets to analyze the cytotoxicity of NK cells against K562 tumor target cells or macrophages. NK cells intracellular cytokine expression (Granulysin, Granzyme B, Perforin, and IFNg) was measured by flow cytometry. In another set of experiments, tumor target K562 cells or macrophage subsets were suspended in complete media (RPMI-1640+10% FCS) and stained with carboxyfluorescein diacetate succinimidyl ester (CFSE), followed by washing. Effector NK cells and target cells were plated together in 96-well plates in an effector-to-target (E:T) ratio of 10:1, 5:1, and 1:1. Effector cell populations were incubated with target cells for 4 h at 37° C. followed by staining with 7-AAD (BD Biosciences, San Jose, Calif.) at 37° C. for 30 min. Following incubation, cells were resuspended and CFSE+/7-AAD+ cells were counted by flow cytometry. A minimum of 25,000 target cells were acquired in 100 μl, which generally yielded 8,000-10,000 CFSE+ events. Background death of target cells alone was determined for each time point and subtracted from all data.

Flow Cytometry

All samples were prepared in triplicates and the mean of the three samples was considered as an individual data. In each replicate, at least 25,000 live leukocytes were acquired. Samples were acquired in either BD LSRILFACSAria IIu or FACS Calibur flow cytometers (BD Biosciences, San Jose, Calif.). Analysis of fluorescence-activated cell sorting (FACS) data was done with FlowJo v. 10.3 software (Tree Star). Analysis of cell population was performed based on a 4-step criteria.

Statistical Analysis

Statistical analysis was done using the SAS MIXED procedure (version 9.3, SAS Institute, Inc., Cary N.C., USA). Data was tested for normality by Kolmogorov-Smirnov test and transformed to natural logarithms or ranks as appropriate when not normally distributed. Comparisons between the two groups were done by Student's t-test or Mann Whitney U test. Categorical data was analyzed with Fisher's exact test and odds ratios (OR) with 95% confidence intervals (95%-CI) was calculated. The significant difference threshold was set at p≤0.05 and p>0.05 to p≤0.10 indicated that a significance was approached. Data is presented as the mean±SEM.

Differentiated Thyroid Cancer (DTC) Risk in Graves Disease

Differentiated thyroid cancer associated with Graves disease is believed to have more aggressive features. As previously stated, the clinical experience in these examples argues against this hypothesis. Here, the characteristics of DTC associated with GD were investigated by reviewing prospectively collected database of patients undergoing thyroidectomy, mostly for thyroid tumors (FIGS. 2A-2D). Data for tumor size, tumor focality, extra-thyroidal extension, lymphatic or distant metastases, and need for reoperation were analyzed. Data for thyroid pathology and antibodies titers against Tg-Abs, TPO-Abs, TR-Abs (also known as TBII and TSI-Abs) were also analyzed.

The relationship of DTC with all autoimmune thyroid diseases (AITD) was analyzed. The data from 2633 consecutive patients, recruited over 19 years (FIG. 2A) was analyzed. On histology, 206 patients were found to have GD, 576 had HT, while the remaining (n=1851) were found not to harbor any autoimmune thyroid disease (non-AITD). Subjects with HT were further subdivided into two subgroups: 211 subjects with hypothyroidism (Hypo-HT) and 365 subjects with normal thyroid function (EHT; all in FIG. 2A). Populations with either GD or HT were compared among themselves for the presence of DTC and with the non-AITD population.

FIG. 11 is Table 3 showing—Differentiated thyroid cancer incidence and pathological features in patients with and without autoimmune thyroid.

Abbreviations: Non-AITD subjects without any form of autoimmune disease by pathology, HT subjects with Hashimoto's thyroiditis, GD subjects with Graves' disease, EHT subjects with Hashimoto's thyroiditis by pathology and normal thyroid function, Hypo-HT subjects with hypothyroidism due to Hashimoto's disease, Macro differentiated thyroid cancer larger than 1 cm in maximum diameter, FVPTC follicular variant of papillary thyroid cancer, Other variants other forms of papillary thyroid cancer, i.e. oncocytic, solid, tall cell variants, Mets distant metastases, I-131 post-operative treatment with at least one dose of I-131. Comparison of thyroid cancer features with Fischer's exact test, between thyroid cancers found in the background of thyroid autoimmunity and those found in the absence of autoimmune thyroid diseases (non-AITD). Odds ratios are reported as a comparison of the proportions between subjects with non-AITD and subjects with autoimmune thyroid disorders. Odds ratios for the presence of DTC are estimated between subgroups of subjects with AITD and subjects without (non-AITD). The odds ratios for DTC are estimated between different subgroups as well. Bold indicates a statistically significant difference between ratios.

The odds ratios (OR) and 95% confidence intervals (95% CI) for having DTC were calculated (FIG. 11 Table 3). A similar comparison was made between subgroups of HT subjects with EHT and Hypo-HT and the results are presented also in FIG. 11—Table 3. It was observed that DTC was less common in GD (FIG. 11—Table 3 and FIG. 2B) and that within the HT group, EHT had a higher proportion of DTC (FIG. 11—Table 3 and FIG. 2C).

The features of tumor aggressiveness of DTC among GD, HT, and non-AITD subjects were then compared, based on tumor histology (presence of follicular thyroid cancer as compared to papillary thyroid cancer, percentage of subjects with follicular variant of papillary thyroid cancer, or aggressive variants of papillary thyroid cancer), tumor size, extra-thyroidal extension of the tumor, lymph node metastasis, distant metastasis, use of I-131 therapy, and need for reoperation. It was observed that GD was associated with less aggressive forms of DTC as compared to HT or non-AITD (FIG. 11—Table 3 and FIG. 2D). HT patients also had milder forms, compared to non-AITD as measured by several, but not all parameters (FIG. 11—Table 3). Lastly, the presence of disease specific-antibodies was checked for. Thyroid stimulating immunoglobulin (TSI) titers were available for 102 subjects with GD: 8 of them with DTC and 94 of them with benign disease. The presence of elevated TSI titers was similar in both, DTC (5/8) and benign disease (60/94), OR 0.94 (95% CI 0.21-4.20, p>0.99). Similarly, data on TR-Abs was available on 45 subjects with GD, 5 subjects with DTC and 40 subjects with benign disease. The presence of elevated titers of TR-Abs was similar in both groups with DTC (4/5) and benign disease (32/40), OR 1.00 (95% CI 0.10-10.2, p>0.99). Data was available on TPO antibodies titers in 136 patients with GD and in 295 patients with HT. For detailed analysis, the subjects were split into two groups: those with high titers of TPO Abs (>=100 IU/L) (TPO+) and those with low or absent titers (<100 IU/L) (TPO−). The frequency of DTC in GD and HT patients as a function of auto-antibody titers was compared. Low/absent TPO titers were associated with a higher risk for DTC both in GD (OR 3.7, 95% CI p=0,043) and HT (OR 2.3, 95% CI 1.4-3.7, p<0.001). This association persisted when these proportions were compared in Hypo-HT (OR 6.5, 95% CI 2.9-14.5, p<0.001) but not in EHT (OR 1.3, 95% CI 0.7-2.5, p=0.43).

Intra-Thyroidal Immune Profiling Revealed Differential NK Cell Activity and Macrophage Polarization

The microenvironment and cellular mechanism of protection from DTC in GD were further investigated. Since failure of tumor immunity allows for cancer growth, it was reasoned that immune profiling of the thyroid gland immune infiltrates may unravel the cause for this failure. The intra-thyroidal mononuclear cell infiltrates in patients with EHT (n=8) and GD (n=8) were compared. The data revealed that NK cells identified as CD3-CD56+ cells were significantly more abundant in GD as compared to EHT (FIG. 3A). The activation status and cytotoxic profile of these NK cells were then compared in both conditions. The NK cells carrying high levels of interferon gamma (IFNg), which reflects their activation status, were significantly higher in numbers in GD as compared to EHT (FIG. 3B). The activated NK cells also produced significantly higher amounts of cytotoxic granules in GD than in EHT including Granulysin, Granzyme B, and Perforin (FIGS. 3C-3D).

Macrophages identified as CD68+ cells were also significantly more abundant in GD as compared to EHT (FIG. 4A). Concentration of B cells (CD19) was not different in either condition (FIG. 4B). Humoral responses (CD19) in EHT and GD were quantitively similar in both diseases (FIG. 4B). By sorting macrophages into M1 and M2 subpopulations, it was found that M1 macrophages present in GD were secreting significantly higher levels of chemokines (CCR2 and CXCR1) as compared to M1 macrophages present in EHT (FIG. 5A). It was also observed that cytokines that characterize the M1 pro-inflammatory phenotype, TNFa and IL-12, were higher in GD than in EHT, significantly for TNFa and with a trend towards statistical significance for IL-12 (FIG. 5A). In contrast, M2 macrophages were significantly more abundant in EHT than in GD as detected by expression of ARGINASE1 and DECTIN1 (FIG. 5C). Also, M2, anti-inflammatory cytokines, and IL10 were significantly higher in EHT than in GD (FIG. 5C).

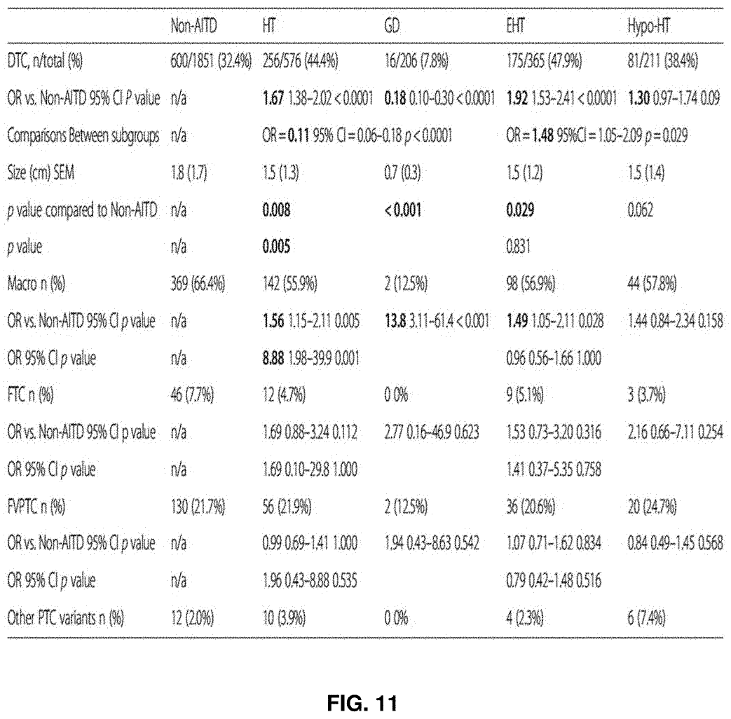

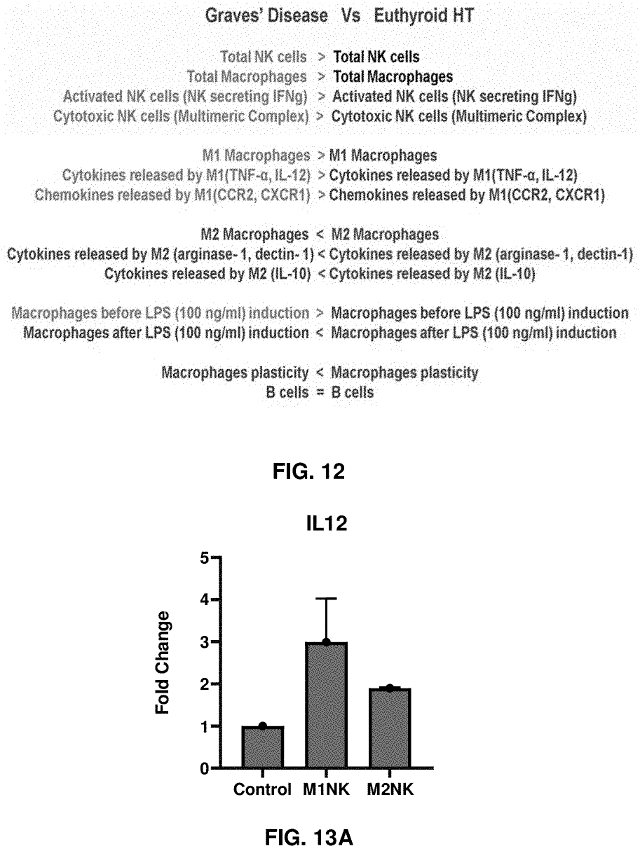

FIG. 12 shows Table 4 of a summary of the immune cell profiling from thyroid bed microenvironment of Graves and Euthyroid HT setting

Next, the intrathyroidal samples from GD and EHT patients were activated (induced) in vitro with high dose of LPS (100 ng/ml) for 54 h. The LPS activation significantly increased the combined expression of M1 phenotype markers (CD68+CCR2, CD68 CXCR1, CD68+IL12, CD68+iNOS and CD68+TNFa) and M2 phenotype markers (CD68+ARGINASE1, CD68+IL10, and CD68+DECTIN1) in EHT; whereas there were non-significant effects on the activated M1 or M2 macrophage phenotypes in GD samples (FIGS. 5B and 5D). In the setting of EHT, M1, and M2 macrophage phenotypes were not only different but also had a higher degree of plasticity as compared to the GD setting (FIGS. 5A-5D). It was also observed that induction of intrathyroidal samples with higher doses of LPS induced macrophage proliferation, which was significantly increased in EHT as compared to GD.

LPS, like IFNg, acts as a pro-inflammatory stimuli for M1 macrophages, and strongly promotes IL-12 mediated T helper 1 responses; but they (M1s) also induce innate anti-tumoral responses through activation of resting NK cells, which eliminate tumor cells and maintain a pro-inflammatory microenvironment. Therefore, NK cells were investigated further.

### Functional Outcomes are Mediated by NK Cell-Macrophage Cross Talk

To dissect the cross talk between NK cells and macrophages in the setting of GD and EHT, the in-vivo microenvironment was re-constituted in vitro. Macrophages were co-cultured with activated NK (NA) and resting NK (N0) cells to mimic, as close as possible, the in-vivo context.

Human peripheral blood monocytes were isolated and differentiated into macrophage subsets of M0, M1 or M2 phenotypes (M1 and M2 structural phenotypes shown in FIGS. 6A-6B), and then co-cultured with autologous NK cells. The proportion of M1 and M2, as well as NK cells in active (NA) or resting (N0) form that were able to be differentiated is shown in FIG. 6C as quantified by flow cytometry.

To determine whether NK cells-derived IFNg is sufficient to polarize macrophages from M2 to M1 phenotype, an autologous co-culture of M1-NA, M2-NA, M1-N0, and M2-N0 was generated. Autologous co-cultures of M2 macrophages with NA/N0 NK cells were stained with M1 phenotype markers. The expression profiles were recorded by using flow cytometry and revealed that autologous co-cultures of M2 with NA/N0 upregulate pro-inflammatory chemokines (CXCR2 and CX3CR1) and cytokines (IL12 and TNFa) in M2 macrophages which were comparable to M1 macrophage expression profiles (FIGS. 6D-6F). Similar results were observed in autologous co-cultures of M1 macrophages with NA/N0 NK cells which were stained for M2 markers (FIGS. 6G-6I). Interestingly, it was also observed that M2 co-cultured with activated NK (NA) cells significantly increases the IFNg expression by NK cells (FIG. 6K) which consequently drives the plastic M2 towards M1 phenotype.

NK Cell Mediated Killing of Autologous Macrophage Subsets

To evaluate the cytotoxic role of NK cells upon autologous macrophage interactions, the different macrophage subsets were co-cultured with NK cells at 1:10 and 1:1 ratio. A reciprocal relationship between NK cell activation status and the macrophages' phenotypic responses was observed. Cytotoxicity was observed towards all three subsets of macrophages (M0, M1, and M2), but cytolysis was significantly higher for M2 macrophages (FIG. 6M). It has been reported that NKP46, an NK cell activating receptor, plays a major role in the killing of M0 and M2 macrophages. NK cells co-cultured with M1 macrophages express a higher level of NKP46 as compared to NK co-cultured with M0 and M2 macrophages. In another experiment, when unpolarized M0 cells were co-cultured with activated NK cells either in presence of M1 or M2 differentiation media, it was observed that activated NK cells promoted the differentiation of M0 to M1 phenotype only. Thus, in the presence of M1 differentiation media, soluble factors activated NK cells which, in turn, favored M1 dominance, as seen in the background of GD. However, in the absence of NK cells, soluble factors favors M0 differentiation towards M2 dominance, as in EHT.

Cytotoxic Activity of NK Cells Against K562 Target Cells

The cytotoxicity of NK cells (effector cells) against K562 tumor cells (target cells) was then tested. It was confirmed that activated NK cells (NA) significantly increased cytotoxic activity against K562 cells as compared to resting NK cells (N0).

Discussion

Inflammation is associated with cancer in most organs. In the thyroid gland, inflammation has been linked to DTC by some. EHT, a silent state of chronic inflammation, is associated with the presence and severity of thyroid cancer. However, controversy remains regarding the potential association of thyroid cancer and its severity in GD. Without wishing to be bound by theory, it is believed that there is an association and even an increased mortality in patients with DTC associated with GD. The clinical observations and accompanying data in these examples argue against this hypothesis, however.

HT and EHT are phenotypically different thyroid autoimmune diseases. In HT, thyroid glands have lymphocytic infiltrates functionally different than those accompanying thyroid cancers in EHT. Lymphocytes present in HT are mainly effector cells while lymphocytes accompanying thyroid cancer in EHT appear to be inactive and under immune regulation. HT is also functionally, different than GD, however both are symptomatic autoimmune diseases of the thyroid.

As with HT, the results show that GD seems to be infrequently associated with thyroid cancer (as opposed to EHT). When thyroid cancer coexists with GD, a less aggressive form of differentiated thyroid cancer is more common. The immune microenvironment of cancer coexisting with GD appears to be more pro-tumor elimination than in patients without GD. The possibility of selection bias is present, however, as a patient with GD may be diagnosed earlier than patients with silent or non-AITD. Nonetheless, the different immune process present in GD and characterized here is consistent with the observed clinical outcome.

The presence of a strong humoral autoimmune response in the form of high TPO-Ah, Tg-Ab, and TR-Ah titers appears to be protective from DTC in patients with GD as judged by the epidemiological observation. To the contrary, the absence of high autoantibody titers appears to confer some risk for DTC in the setting of GD. TSI however were equally present. TSI have the ability to engage the TSH receptor and induce hyperthyroidism, which is clinically associated with benign outcomes.

It is also demonstrated here that cellular immune infiltrates accompanying thyroid cancer in the background of GD have higher proportions of NK cells and significantly higher numbers of M1 macrophages as compared with EHT. On the other hand, the immune infiltrates in EHT are low in NK cells and M1 macrophages, but have a higher M2 macrophage presence. In particular, it was noticed that the NK cell population in GD differs from the one in EHT, in that NK cells in GD are mostly activated. Measurements of cytotoxic multimeric complexes of activated NK cells (Granulysin, Granzyme B, and Perforin) as well as INFg were all high in the GD immune infiltrates. Despite the differences in the humoral responses between GD and EHT noted above, the quantification of B cells (CD19) was not different in either group.

As mentioned above, macrophages were more abundant in lymphocytic infiltrates of GD than of EHT patients. Although disproportionally, induction of the intra-thyroidal immune infiltrates with lipopolysaccharide (LPS) increased the number of macrophages in both GD and EHT. It is known that the stimulation of resting macrophages (M0) with Th1 cytokines (i.e., IFNg) or TLR4 ligands (i.e., LPS) induces the classical polarization towards M1, which displays strong microbicidal and tumoricidal properties and preferentially promotes inflammatory responses. In contrast, the alternative polarization towards M2 macrophages is induced by the Th2 cytokine IL-4. Specifically, M1 macrophages were significantly more abundant in the background of GD while M2 were significantly more abundant in the background of EHT. Both M1 and M2 phenotypic markers increased post-induction in EHT, but induction had a minimal effect on the macrophages in GD, indicative of higher degree of plasticity of macrophages in the background of EHT.

M1 (but not M0 or M2) macrophages can activate NK cells. Soluble mediators as well as cell-to-cell interactions of M1 with NK cells activate the later into its cytotoxic state. Co-culturing NK activated (NA) or resting (N0) cells with M2 macrophages demonstrated distinct phenotypic outcomes. Co-culturing NA/N0 cells with M2 macrophages increased the expression of M1 phenotype surface chemokines (CCR2, CX3CR1), and intracellular cytokines (IL-12, TNFa). However, an interesting phenomenon was observed. Co-culturing activated NK cells (NA) with M2 macrophages significantly upregulated the IFNg expression of NK cells (FIG. 6K). The change in the expression profile from M2 to M1 phenotype and the expression of IFNg by NK cells was inter-related, which may be explained as NA and M2 co-cultures upregulate CD80, sustaining Th1 responses, and CD48, a major ligand for 2B4, both expressed by M2 macrophages during co-culture.

2B4, an NK cell activating receptor, binds with CD48 on macrophages and triggers NK cell cytotoxicity by enhancing degranulation and release of IFNg, which in turn sustains CD48 expression by M2 macrophages. The more aggressive response of NK cells, when co-cultured with M2, apparently resulted from significant NK-cell expressed IFNg, which upregulated pro-inflammatory cytokines (IL-15/IL-15Rα) complex, and IL-1β by M2 macrophages. It is quite interesting that the M1 macrophages were tolerant towards second IFNg stimulation in terms of IL-1β transcription, whereas M2 macrophages were strongly upregulated in first stimulation and equally responsive to second IFNg stimulation which potentially transcribes higher levels of IL-1β than in M1. Although has pro-inflammatory effects, it also actively participates in NK cell activation as IL-1β upregulates NK cell activating receptor NKp44. Therefore, the IFNg dominant microenvironment favored the M2 macrophages polarization towards M1.

M2 macrophages were observed to respond better to IFNg as compare to M1. The same events have been observed in GD, where highly activated NK cells created a pro-inflammatory M microenvironment as opposed to the EHT condition where M2 non-inflammatory dominance showed a significantly higher degree of macrophage plasticity after induction with LPS. These experiments explain how in natural disease conditions, macrophage plasticity is microenvironment/NK-dominance dependent. The M2 macrophages' plasticity is exploited by using TLR4 agonist (LPS in this example) which has the capacity to create a pro-inflammatory microenvironment and re-educate the M2 macrophages towards M1 phenotype.

The increase in cytotoxicity of NK cells exposed to M1-differentiation media may be because of M1 macrophages upregulating CD48, which is a major ligand for 2B4 (expressed by NK cells), so NK-M1 co-culture modulated by 2B4-CD48 interaction may increase the NK cells IFNg expression. Therefore, the production of IFNg may act as a major inductor of NK cell cytotoxicity towards tumor target cells. Thus, in the setting of NK activation, the final differentiation outcome is M1 dominance, as seen in the background of GD. Moreover, the M2 dominance, as seen in EHT, seems to affect recruitment of NK cells at the cancer site.

Therefore, the tumor microenvironment plays a significant role in limiting the activation of NK cells that can kill tumor cells through IFNg. M2 macrophages are modulated to the M1 phenotype and such activation can cause tumor recession. M1 macrophages activate NK cells and induce IFNg production which in turn plays a key role in inducing M1 polarization. Remarkably, the data in this example shows that M2 co-cultured with NK cells reverts an established M2 phenotype to its M1 cytotoxic potential.

Although this example is focused on DTC, the most malignant form of thyroid cancer (anaplastic) develops within a matrix of macrophages. Anaplastic thyroid cancer distinctively grows in a very dense network of interconnected macrophages (flagellating like M2 s as in FIG. 6B) in direct contact with intermingled cancer cells. Even anaplastic thyroid cancer metastasis depends on local macrophages for establishment and progression.

### Example III

Crosstalk Between NK Cells and Macrophages in Thyroid Cancer Coexisting with Graves' Disease and Euthyroid Hashimoto's Thyroiditis

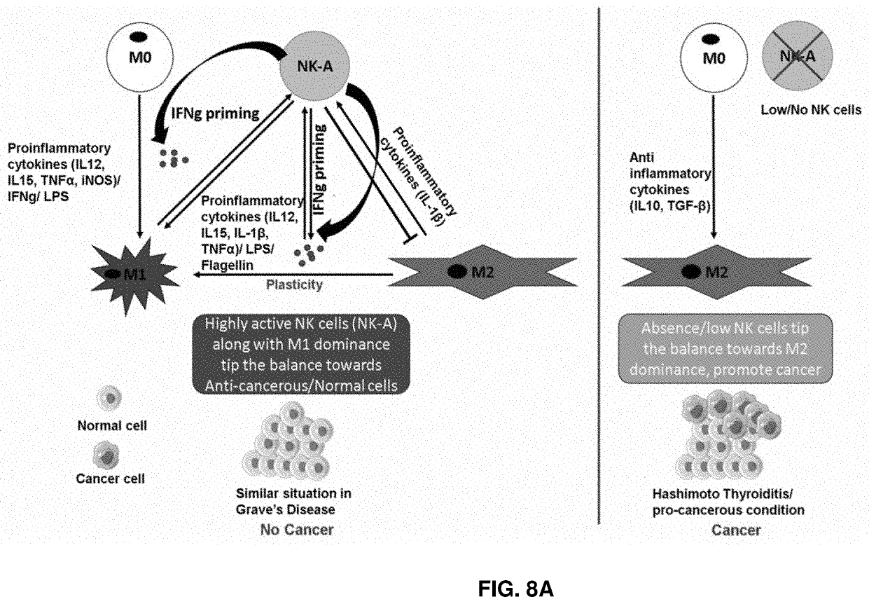

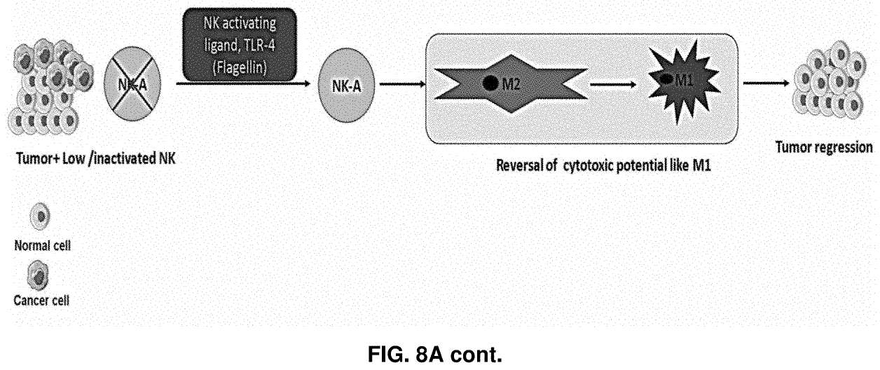

FIGS. 8A-8B provide illustration showing crosstalk between NK cells and macrophages in thyroid cancer coexisting with Graves' disease and euthyroid Hashimoto's thyroiditis. Activated NK cells and M1 macrophages induce tumor regression and protect from cancer as in case of Graves' disease (No cancer) while euthyroid HT, low/no NK cells tip the macrophages balance towards M2-dominance may contribute to tumor progression (pro cancerous/cancer) (FIG. 8A). Illustration showing the immunomodulatory role of flagellin, a therapeutic approach for anaplastic thyroid cancers which are densely intermingled with ramified M2 macrophages with long and thin cytoplasmic processes (FIG. 8B).

### Example IV

Flagellin Induces the Plasticity of Macrophages from M2 to M1

Bacterial flagellin is a major structural component of flagellum, locomotory organ present mostly in all gram negative and few gram positive bacteria. Due to its occurrence in wide range of bacterial species and high abundance in bacterial cell, flagellin form compelling pathogen associated molecular pattern (PAMP) and chief target for various immune cells.

Flagellin consists of highly conserved N-terminal and C-terminal domains (D1 and D2 domain) across the species contain primarily α-helical structure. Additional hypervariable intervening domain (D3 domain) chiefly consists of β-sheet structure. Conserved domains (D1 and D2 domain) are essential for binding of flagellin to TLR-5 and are sufficient for eliciting pro-inflammatory response. Flagellin extracellularly binds to the TLR-5, culminates in the activation of transcription factors like AP-1 and NF-κB through myd-88 dependent pathway.

In fact, activation results in induction and secretion of pro-inflammatory cytokines, chemokines and other mediators required for the effective immune system. Inside the cell, flagellin engage with the NOD like receptor NLRC4, mediates the activation of caspase-1, which finally cleaves pro-interleukin-1β into active IL-1β, a potent pro-inflammatory cytokine that is critical for host responses to infection and injury.

Flagellin is a virulence factor required for the adhesion and invasion of the bacteria. Nevertheless, it is used as a potent immune-stimulant, which activates the immune cells. Flagellin is useful as adjuvant for many potent vaccines, which proven very safe to use therapeutically. Moreover, flagellin also exercises anti-tumor and radioprotective activities, and has demonstrated exceptional uses to ameliorate the tumor growth and radiation induced tissue damage.

This example shows a significant use of flagellin, where flagellin induces the plasticity of macrophages from M2 to M1 under in vitro and in-vivo conditions.

The reversibility of polarization has a decisive therapeutic value, especially in diseases in which an M1/M2 imbalance plays a pathogenic role.

Described herein is a cancer immunotherapy for thyroid cancer with an M2 macrophage-dominant microenvironment in case of Anaplastic thyroid cancer which are architecturally dense intermingled ramified M2 macrophages with long and thin cytoplasmic processes. Activation of NK cells by using TLR ligands (flagellin) disrupts the balance of macrophages toward the M1 phenotypes.

But, more important than the M2 association with thyroid cancer is the discovery that activated NK cells can drive M1 differentiation, which in turn is cytotoxic to cancer cells and down-regulates 11/12s. Another TLR ligand, for example, induces activation of NK cells and has less adverse effects compared with LPS. Therefore, immunomodulatory flagellin is an alternative treatment option for cancers dependent on M2 support (such as anaplastic thyroid cancer).

In this example, thyroid cancer behavior in the background of GD is shown to depend on the NK-M1 dynamics. Cancer is capable of disrupting the M1 dominance by tipping the macrophage plastic balance towards the M2 phenotype. Fortunately, this process is therapeutically reversible, in that TLR5 agonist (flagellin for example) may reverse the M2 phenotype.

In the setting of EHT, the macrophage phenotype was not just different but also had a higher degree of plasticity than in the GD setting. In GD, the presence of functionally active NK cells and higher M1/M2 macrophage ratio may provide Graves' patients with a more effective form of tumor immunity. However, in the absence/low count of active NK cells, macrophage plasticity allows M0 to differentiate to the M2 phenotype, which may explain the higher risk of thyroid cancer in EHT.

Overall, studying the tumor microenvironment in thyroid cancer enables an understanding of the different clinical behaviors of thyroid cancers with similar histological and molecular signatures. Moreover, clinical behavior of other cancers may likely be related to these specific immune cell players. In the era of cancer immunotherapy, knowledge about tumor immunity is no longer basic but clinical.

The findings herein demonstrated that DN T cells regulate proliferation and effector function of T cells. Fas-FasL leads to activation-induced cell death, a form of apoptosis induced by repeated TCR stimulation, responsible for the peripheral deletion of activated T cells. Fas-FasL, naturally resistant DN T cells also induce apoptosis to NK cells. Absence of, or a low count of, active NK cells, macrophage plasticity allows macrophage subtype M0 to differentiate to the M2 phenotype (precancerous/cancerous).

To determine whether NK cells-derived IFNg is sufficient to polarize macrophages from M2 to M1 phenotype, an autologous co-culture of M0-NA, M1-NA, M2-NA, M0, M1, and M2 was generated. Autologous co-cultures of M0/M1/M2 macrophages with NK cells were subjected to total RNA isolation. One μg of RNA was converted into cDNA using iScript Reverse Transcription kit.

Methods

IL-1, IL-4, IL-6, IL-10, IL-12, IL-13, IL-15, IL-18, IL-23, IL-27, TNF-α, TGF-β, IFNg, CCL1, CCL2, CXCL10, ACTB human gene expression was quantified using SYBR Green chemistry (Applied BioSystems, Life technologies) with specific human primers. The expression was quantified using the ΔΔCT method with 18 S RNA as the endogenous control. Minus-reverse transcriptase samples were used as negative controls to test for DNA contamination. The expression profiles were recorded.

Results

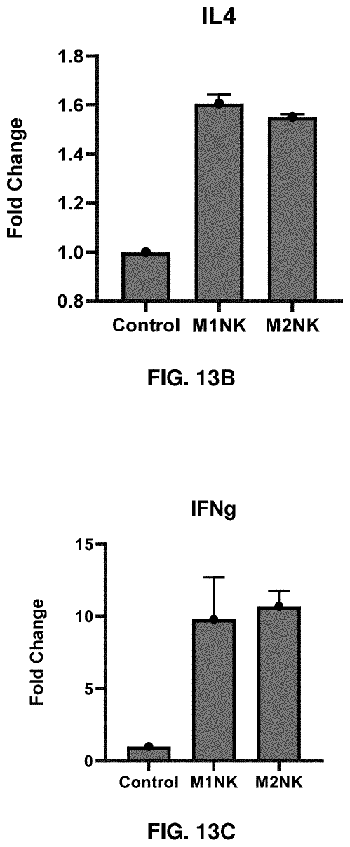

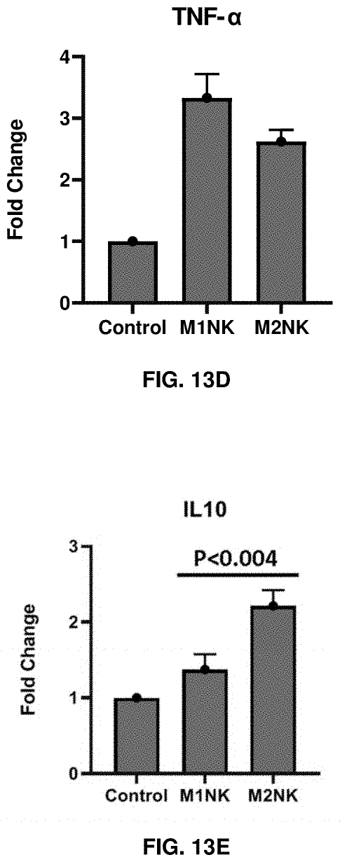

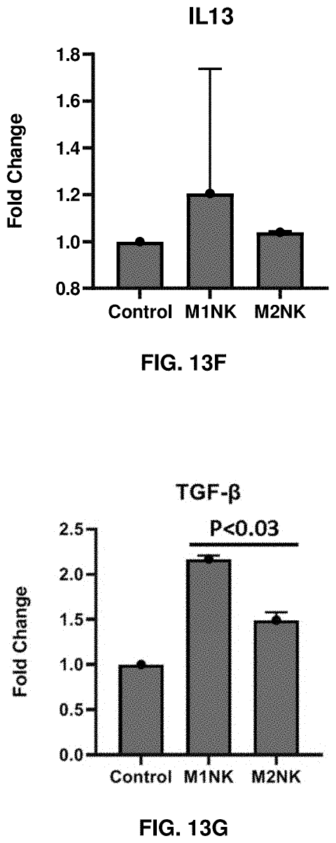

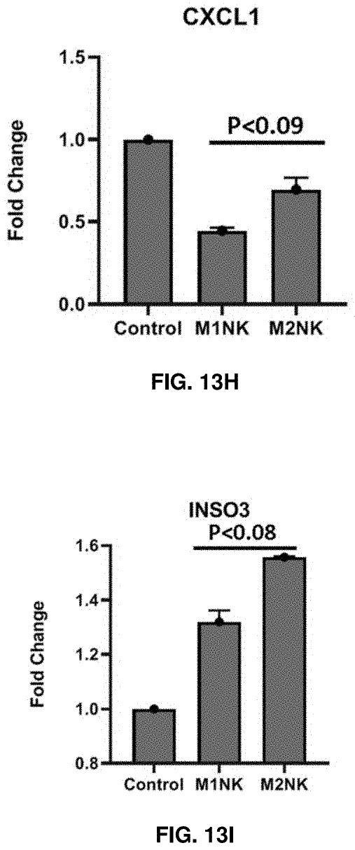

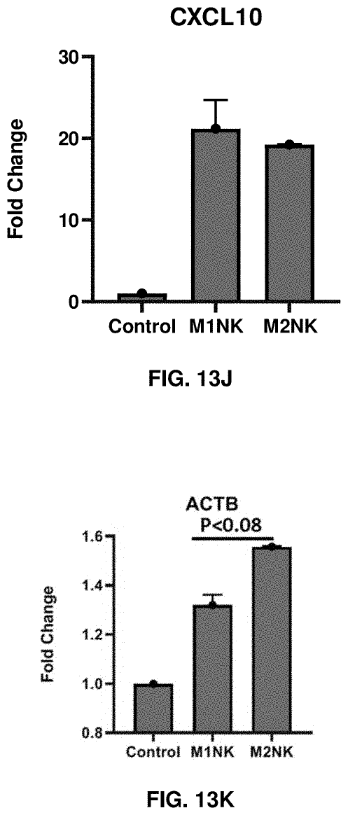

Proinflammatory cytokines expression of M2 macrophages were comparable to M1 macrophage expression profiles (FIGS. 13A-13K).

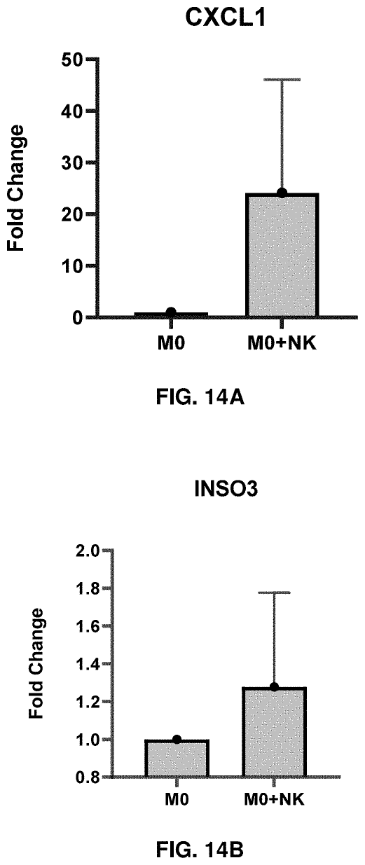

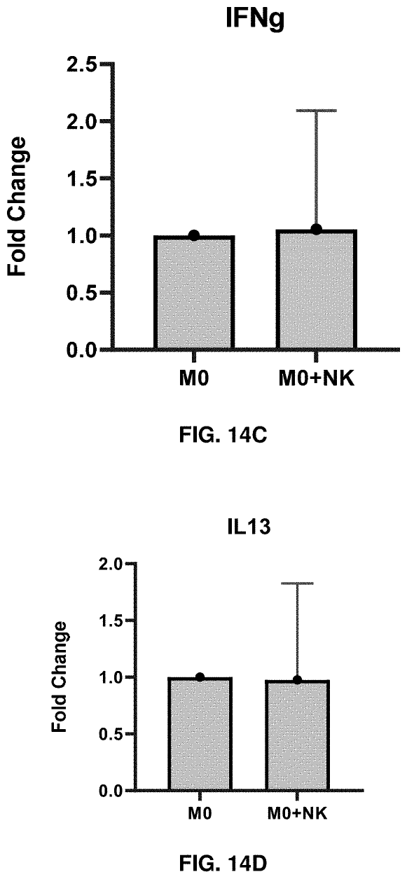

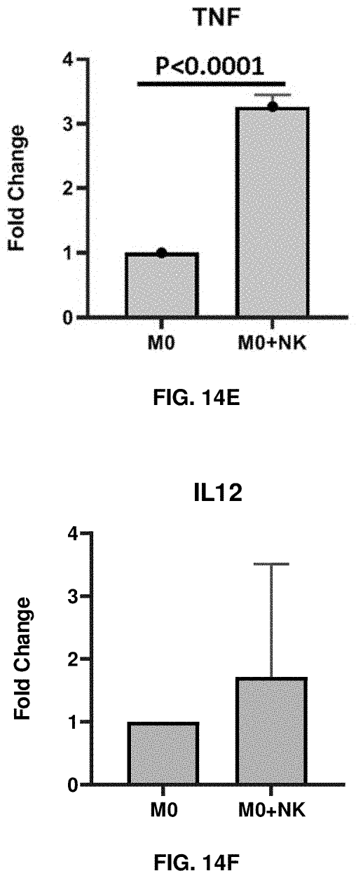

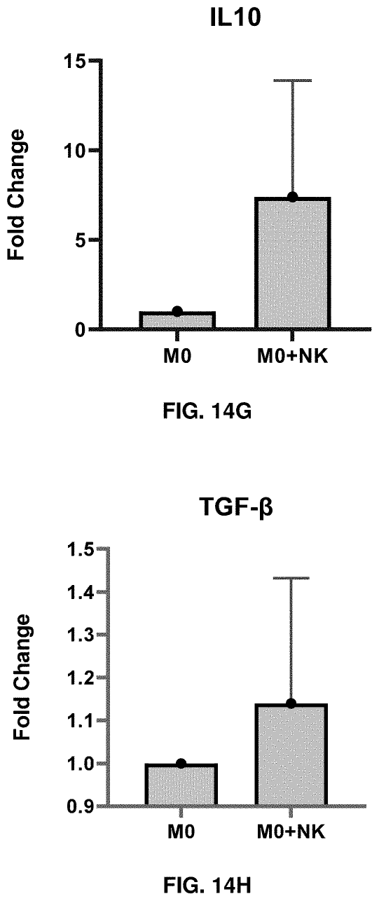

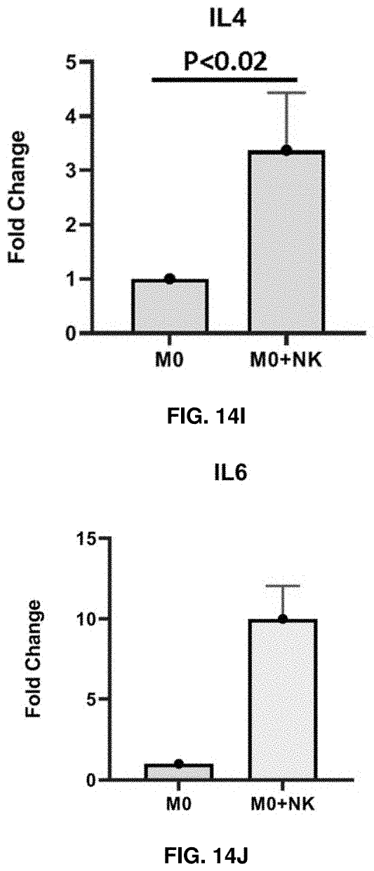

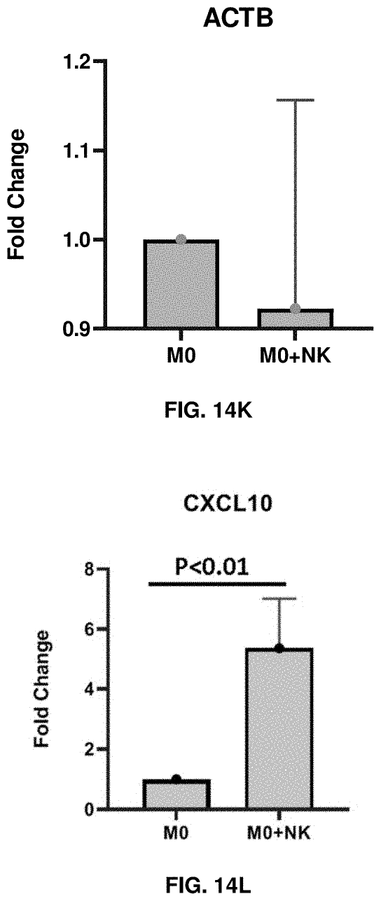

Similar results were observed in autologous co-cultures of M0 monocytes with NK cells (FIGS. 14A-14L). Interestingly, it was also observed that M0 co-cultured with activated NK (NA) cells significantly increases the proinflammatory cytokines and chemokines expression which consequently drives the M0 monocyte differentiation towards M1 phenotype.

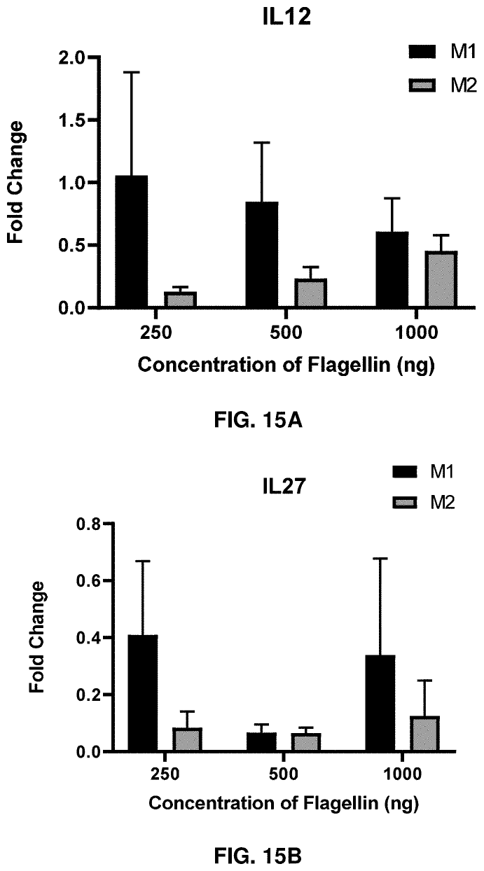

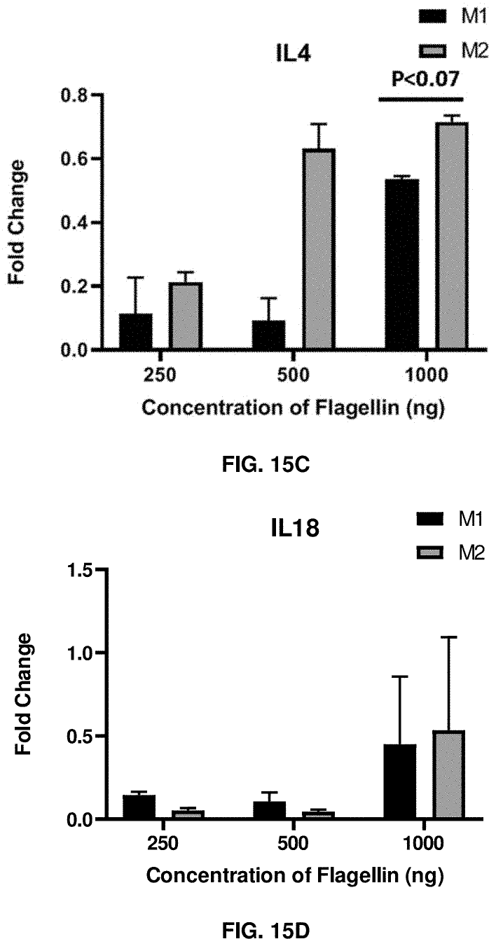

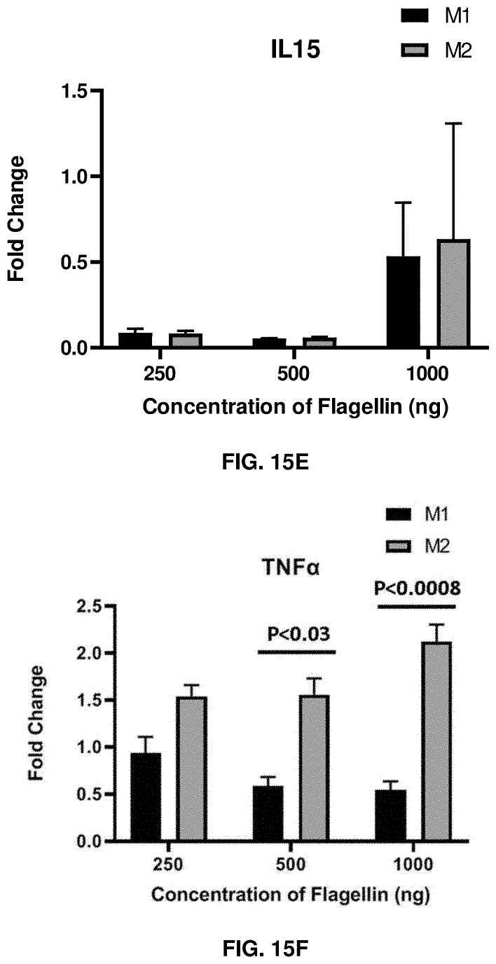

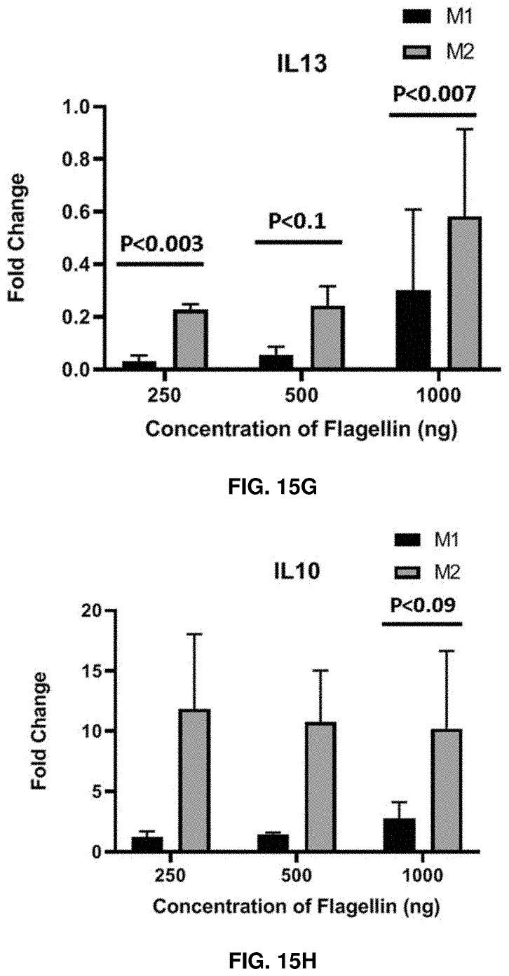

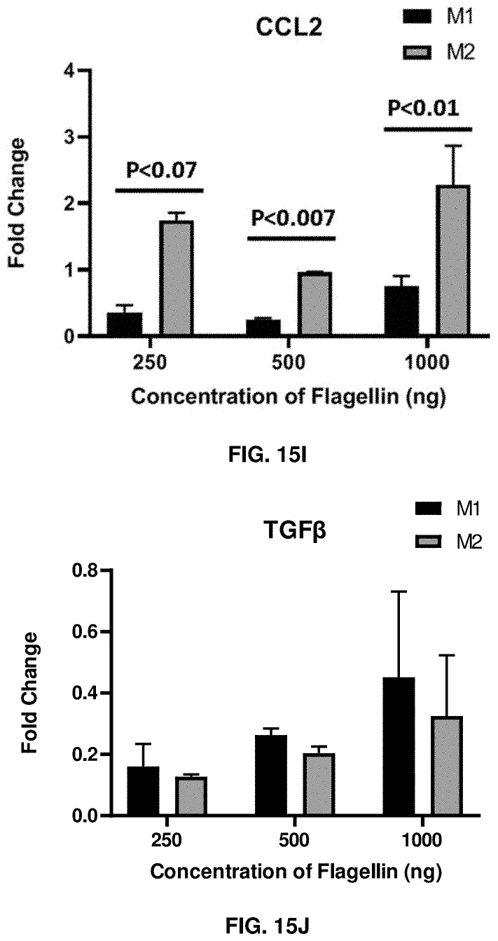

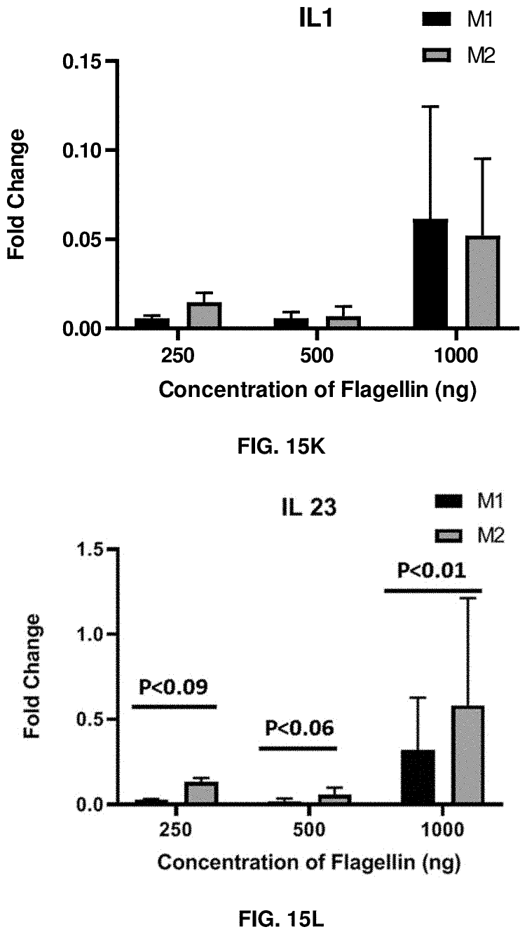

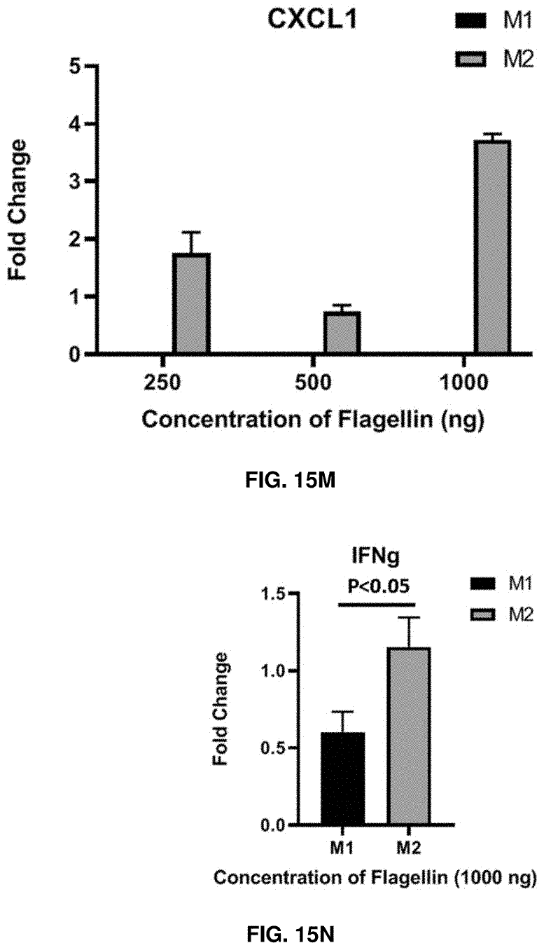

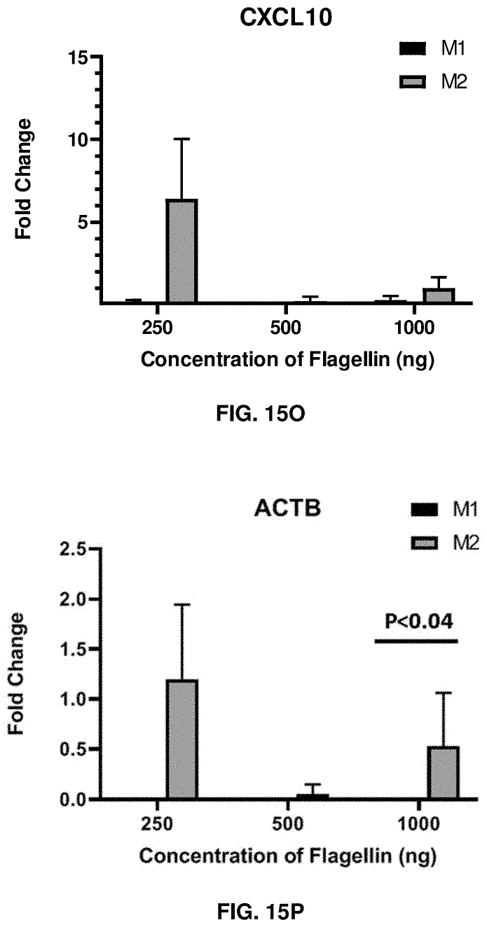

To determine whether TLR-5 agonist flagellin is capable to polarize macrophages from M2 to M1 phenotype as like NK cells or LPS treatment. An autologous co-culture of M1—with 250 ng Flagellin, M1—with 500 ng Flagellin, M1—with 1000 ng Flagellin and similarly M2—with 250 ng Flagellin, M2—with 500 ng Flagellin, M2—with 1000 ng Flagellin was generated. Autologous co-cultures of M1/M2 macrophages with 250, 500 and 1000 ng flagellin were subjected to total RNA isolation. One μg of RNA was converted into cDNA using iScript Reverse Transcription kit. IL-1, IL-4, IL-6, IL-10, IL-12, IL-13, IL-15, IL-18, IL-23, IL-27, TNF-α, TGF-β, IFNg, CCL1, CCL2, CXCL10, ACTB human gene expression was quantified using SYBR Green chemistry (Applied BioSystenis, Life technologies) with specific human primers. The expression was quantified using the ΔΔCT method with 18 S RNA as the endogenous control. Minus-reverse transcriptase samples were used as negative controls to test for DNA contamination, Proinflammatory cytokines expression of M2 macrophages were comparable to M1 macrophage expression profiles (FIGS. 15A-15P).

Interestingly, it was also observed that M2 co-cultured with flagellin @ 1000 ng significantly increases the proinflammatory cytokines and chemokines expression which consequently drives the M2 macrophages into M1 phenotype as activated NK cells did in in-vivo condition.

Flagellin @ 1000 ng induces IL12, IL27 expression on M2 macrophages co-cultured and are comparable to M1 macrophages.

IL-1 is a proinflammatory cytokines and Flagellin induces the expression of IL-1 at M2 macrophages and it's comparable to M1 macrophages.

IL-23 is a member of the IL-12 family with pro-inflammatory properties and co-culture with Flagellin induces plasticity to M2 macrophages towards pro-inflammation. Flagellin induces IL-23 expression on M2 macrophages and was comparable to M1 macrophages.

IL-18 is also known as IFNg inducer factor. IL-18 belongs to the IL-1 superfamily and is mainly produced by macrophages. IL-18 is a proinflammatory cytokine that facilitates type 1 responses. Combination of IL-12 and IL-18 induces cell-mediated immunity following bacterial infection like lipopolysaccharide (LPS). IL-18 in combination with IL-12 acts on CD4, CD8 T cells and NK cells to induce IFNg production, consecutively activate macrophages or other pro-inflammatory cells. Flagellin (1000 ng) induces IL-18 expression comparable to M1 macrophages, consecutively simulate IL-12 expression and act as an IFNg inducer. Similarly, IL-15 is a soluble (s) IL-15/IL-15Rα complexes secreted by macrophages, regulate tumor-infiltrating NK cells and lymphocyte numbers. Flagellin (1000 ng) induces IL-15 expression in M2 comparable to M1 macrophages.

TNF-α is a pro-inflammatory cytokines. The primary role of TNF is the regulation of immune inflammation and to inhibit tumorigenesis. Flagellin at the rate of 500 ng and 1000 ng significantly induces TNF-α expression in M2 co-culture and are comparable to M1 macrophages.

IL-13 is an immunoregulatory cytokine, regulates the function of human monocytes and B cells. IL-13 downregulation effects on the production of pro-inflammatory cytokines, particularly of IL-12 by monocytes. Flagellin at the rate of 250 ng and 1000 ng significantly induces IL-13 expression in M2 co-culture and are comparable to M1 macrophages.

Flagellin at the rate of 250 ng and 1000 ng does not have any effect on TGF-β expression.

Chemokine (C-C motif) ligand 2 (CCL2) is also referred to as monocyte chemoattractant protein 1 (MCP1) recruits monocytes at the sites of inflammation. Flagellin at the rate of 250 ng, 500 ng and 1000 ng significantly induces CCL2 expression in M2 macrophages co-cultured with flagellin and are comparable to M1 macrophages.

Flagellin has no effect on CXCL1 expression, it's mainly expressed on TAM.

C-X-C motif chemokine ligand 10 (CXCL10) also known as Interferon gamma-induced protein 10 stimulate pro-inflammatory cytokine production in Macrophages and T helper cells. Flagellin co-culture with M2 macrophages significantly induces CXCL10 at the dose of 250, 500 and 1000 ng.

Certain embodiments of the methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

1. A method for collecting and analyzing a sample, the method comprising:
extracting a tissue sample from a patient through a fine needle aspiration (FNA), wherein the tissue sample is extracted from an area comprising fluid within and adjacent to a thyroid nodule;
analyzing lymphocytes in the extracted tissue sample; and
determining an amount of double negative T cells present in the lymphocytes.

2. The method of claim 1, wherein the FNA is ultrasound-guided FNA.

3. The method of claim 1, wherein the area is where the fluid meets the thyroid nodule.

4. The method of claim 1, wherein the fluid comprises blood.

5. The method of claim 1, wherein the fluid comprises immune cells.

6. The method of claim 1, further comprising analyzing the extracted tissue sample for macrophages and NK cells.

7. The method of claim 1, further comprising removing the patient's thyroid if the thyroid nodule is determined to be, or likely to become, cancerous based on the amount of double negative T cells present in the lymphocytes in the extracted tissue sample.

8. The method of claim 1, further comprising profiling the lymphocytes with flow cytometry.

9. The method of claim 1, further comprising analyzing the extracted tissue sample for chemokines and cytokines using a cytometric bead array.

10. The method of claim 1, further comprising sequencing RNA in the extracted tissue sample.

11. A method for diagnosing thyroid cancer, the method comprising:
obtaining a tissue sample from an area within and adjacent to a thyroid nodule in a patient;
measuring an amount of double negative T cells in the tissue sample relative to other lymphocytes in the tissue sample;
determining whether the thyroid nodule is, or is likely to become, cancerous, wherein a double negative T cell content of greater than 15% in the obtained sample indicates the thyroid nodule is, or is likely to become, cancerous, and a double negative T cell content less than 5% in the obtained sample indicates the thyroid nodule is not, and is not likely to become, cancerous; and
removing the patient's thyroid if the thyroid nodule is determined to be, or likely to become, cancerous; or
not removing the patient's thyroid if the thyroid nodule is not determined to be, or not likely to become, cancerous.

12. The method of claim 11, further comprising administering a treatment based on the determination of whether the thyroid nodule is, or is likely to become, cancerous.

13. The method of claim 11, wherein the tissue sample is obtained through a fine needle aspiration (FNA).

14. The method of claim 11, wherein the tissue sample is obtained through an ultrasound-guided fine needle aspiration.

15. The method of claim 11, wherein the area comprises fluid.

16. The method of claim 15, wherein the area is where the fluid meets the thyroid nodule.

17. A method for diagnosing thyroid cancer, the method comprising:
extracting a tissue sample from a patient through a fine needle aspiration (FNA), wherein the tissue sample is from an area comprising fluid within and adjacent to a thyroid nodule, wherein the fluid comprises immune cells;
measuring an amount of double negative T cells, macrophages, and NK cells in the tissue sample; and
determining whether the thyroid nodule is, or is likely to become, cancerous;
wherein a double negative T cell content of greater than 15% in the extracted tissue sample indicates the thyroid nodule is, or is likely to become, cancerous, and a double negative T cell content less than 5% in the extracted tissue sample indicates the thyroid nodule is not, and is not likely to become, cancerous.

18. The method of claim 17, wherein the FNA is ultrasound-guided FNA.

19. The method of claim 17, further comprising removing the patient's thyroid if the thyroid nodule is determined to be, or likely to become, cancerous.

20-21. (canceled)
